(19) United States
(12) Patent Application Publication
(10) Pub. No.: US 2024/0241121 A1
(43) Pub. Date: Jul. 18, 2024

(54) High Sensitivity Analyte Network Detection Flow Assays And Related Methods

(71) Applicant: Senzo Health Limited, London (GB)

(72) Inventors: Aron Rachamim, Basel (CH); Jebin Jacob John, London (GB); Mohamed Lemine Dit Youba Diakite, London (GB); Jacob Raby, London (GB)

(21) Appl. No.: 18/412,418

(22) Filed: Jan. 12, 2024

Related U.S. Application Data
(63) Continuation of application No. PCT/IB2022/000468, filed on Aug. 11, 2022.
(60) Provisional application No. 61/232,150, filed on Aug. 7, 2009.
(60) Provisional application No. 63/321,083, filed on Mar. 17, 2022.

(51) Int. Cl. G01N 33/569; G01N 33/573

(52) U.S. Cl. CPC G01N 33/56983; G01N 33/573; G01N 2333/135; G01N 2333/165; G01N 2333/988; G01N 2469/10

(57) ABSTRACT

Articles (e.g., lateral flow assays) and methods for the detection of an analyte are generally described. The assays may involve the use of a network which blocks or restricts flow in the assay, e.g., due to formation of an interconnect network or lattice.

### RELATED APPLICATIONS

This application is a continuation of International Application No. PCT/IB2022/000468; which claims the benefit of U.S. Provisional Application No. 63/232,150, and U.S. Provisional Application No. 63/321,083, the entire contents of each of the aforementioned applications being incorporated by reference herein.

### TECHNICAL FIELD

Articles (e.g., lateral flow assays) and methods for the detection of an analyte on a lateral flow assay are generally described.

### BACKGROUND

The rapid analysis of biological and chemical targets can be important for preliminary or emergency medical screening and this explains the wide use of certain rapid tests for performing the analysis. Many such rapid tests are based on chromatographic techniques using lateral flow assays on paper. Some of these lateral flow devices are generally based on nanoparticles and offer only limited sensitivity. Despite their limitation in sensitivity, these nanoparticle-based tests are preferred because of their simplicity and low production cost. However, improved later flow assays with increased sensitivity are desired.

### SUMMARY

Articles (e.g., flow assays, lateral flow assays) and methods for the detection of an analyte within an interconnected network (e.g., a lattice) are generally described. The subject matter of the present disclosure involves, in some cases, interrelated products, alternative solutions to a particular problem, and/or a plurality of different uses of one or more systems and/or articles.

In one aspect, a flow assay is described, the flow assay comprising a substrate having an upstream position and a downstream position and a binding region comprising a plurality of capture reagents positioned at the downstream position, wherein the flow of at least a portion of the plurality of capture reagents is restricted when an analyte binds to at least a portion of the capture reagents.

In another aspect, a method for detecting a plurality of analytes in a sample using a flow assay, the method comprising, introducing the sample to an upstream position of substrate, wherein the substrate comprises a plurality of capture reagents positioned at a binding region at a downstream position, flowing the sample from the upstream position towards the downstream position, allowing at least a portion of the plurality of analytes of the sample to bind with at least a portion of the plurality of capture reagents, restricting a flow along the flow assay after at least a portion of the plurality of analytes of the sample bind with at least a portion of the plurality of capture reagents, detecting at least one of the analytes.

In another aspect, a flow assay is described comprising a substrate having an upstream position and a downstream position and a binding region comprising a plurality of capture reagents positioned at the downstream position, wherein the plurality of capture reagents is configured to form an interconnected network with a plurality of analytes, the interconnected network comprising a mixture of the plurality of capture reagents and the plurality of analytes interconnected with one another.

In another aspect, a flow assay is described comprising a substrate having an upstream position and a downstream position; a binding region comprising a plurality of capture reagents positioned at the downstream position; and an interconnected network at the downstream position or between the upstream position and the downstream position, wherein the interconnected network comprises a mixture of the plurality of capture reagents and the plurality of analytes interconnected with one another.

In another aspect, a method for detecting a plurality of analytes in a sample using a flow assay is described, the method comprising introducing the sample at an upstream position of a substrate, wherein the substrate comprises a plurality of capture reagents positioned at a binding region at a downstream position; flowing the sample from the upstream position towards the downstream position; flowing a plurality of detection reagents from the upstream position towards the downstream position; allowing the plurality of analytes of the sample to bind with at least some of the plurality of capture reagents; forming an interconnected network comprising a mixture of the plurality of capture reagents and the plurality of analytes; and detecting at least one the analytes.

In another aspect, a flow assay is described comprising a substrate having an upstream position and a downstream position; a binding region comprising a plurality of capture reagents positioned at the downstream position; a plurality of detection reagents positioned upstream of the plurality of capture reagents; and a cassette enclosing at least a portion of the substrate, wherein the cassette comprises a first portion and a second portion opposing the first portion, wherein the substrate is positioned between the first and second portions, wherein the second portion comprises a protrusion extending towards the first portion, and wherein the protrusion presses the substrate against the first portion.

Other advantages and novel features of the present disclosure will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1C schematically illustrate the detection of an analyte using a flow assay, according to some embodiments;

FIGS. 1D-1F schematically illustrate the detection of a plurality of analytes within an interconnected network using a flow assay, according to some embodiments;

FIGS. 1G-1L schematically illustrate interconnected networks comprising at least one analyte and at least one capture reagent, according to some embodiments;

FIGS. 2A-2B are schematic depictions of a cassette that includes a protrusion that can press a substrate against a first portion of the cassette, according to some embodiments;

FIGS. 2C-2F are schematic views of a cassette and its included protrusion, according to some embodiments;

FIG. 3 is photographic image of a substrate, according to one set of embodiments;

FIG. 4 shows photographic images of a substrate cut into small piece, according to some embodiments;

FIG. 5 is a photographic image of small pieces of cut substrate within a 96-well plate, according to some embodiments;

FIG. 6 is a bar chart indicating the absorbance of various piece of the cut substrate, according to some embodiments;

FIG. 7 is a photographic image of two lateral flow assays after use, comparing the flow profile of the two assays, according to some embodiments;

FIG. 8 shows photographic images of lateral flow assays comparing the use of different types and sizes of antibodies as capture reagents, according to some embodiments;

FIGS. 9-10 are images using an antigen from respiratory syncytial virus (RSV) as the analyte, according to some embodiments;

FIG. 11 shows time lapse microscope images of a strip with fluorescently labeled antibodies during a lateral flow assay run, comparing a positive and negative sample to determine the flow properties of the antibody, according to some embodiments;

FIG. 12 shows time lapse microscope images of the labeled enzyme detection reagent during flow through the membrane for positive and negative samples, according to some embodiments;

FIG. 13 shows fluorescent imaging of the strip for a positive and negative sample before and after TMB addition, according to some embodiments; and

FIG. 14 is an image of a strip following TMB addition and development for a positive and negative sample, according to some embodiments.

### DETAILED DESCRIPTION

The present disclosure describes flow assays and methods for the detection of an analyte (e.g., within an interconnected network, a precipitate, and/or a lattice). In some embodiments, flow on the lateral flow assay is reduced (e.g., restricted), for example, when an analyte binds to a reagent (e.g., a capture reagent, a capture antibody) within a binding region. Advantageously, reducing of the flow rate (e.g., when the analyte binds to a capture reagent) may improve in detection of the analyte relative to conventional lateral flow assays where flow of the analyte is not reduced upon binding of the analyte to a capture reagent. In some instances, the flow assay is a lateral flow assay that flows the analyte on a surface of a substrate, such a paper substrate. Many existing lateral flow assays are based on chromatographic techniques on paper substrates and may use, for example, nanoparticles to visually detect an analyte, if present within a sample to be analyzed. However, these colorimetric techniques provide only limited sensitivity. By contrast, enzymatic techniques, such as ELISA (enzyme-linked immunosorbent assay), may provide a higher sensitivity relative to most existing lateral flow assays because ELISA uses an enzymatic reaction and the addition of an enzyme substrate, which can improve the sensitivity of the assay. However, the ELISA process may be prohibitively time consuming and may require more complex processing relative to lateral flow assays.

It has been discovered, within the context of this disclosure, that the sensitivity and the performance of lateral flow assays can be improved by replacing nanoparticles used in traditional lateral flow assays with an enzyme (i.e., a capture reagent) and a chromogenic substrate (i.e., a detection reagent) for the enzyme. By using a chromogenic enzyme substrate in combination with features described herein, it was unexpectedly discovered that the sensitivity of the lateral flow assay could be dramatically improved relative to many existing lateral flow assays. Without wishing to be bound by any particular theory, it is believed the improved sensitivity is achieved at least in part by the formation of an interconnected network (e.g., a lattice, a precipitate) including a capture reagent, and, optionally a detection reagent, and it is believed that the formation of this interconnected network may increase the limit of detection of the assay, resulting in improved sensitivity. It should be understood that the articles and methods described herein may be applied to any suitable flow assays, including microfluidic channel systems, and the aspects described herein are not limited to lateral flow assays.

In an exemplary embodiment, a sample suspected of containing an analyte (or some other species of interest) is deposited onto an upstream position of a substrate, such as a strip of nitrocellulose paper, wherein the strip includes a detection reagent, such as an enzyme, at an upstream position and a capture reagent, such as a capture antibody, at downstream position. The strip may include a wicking material that flows the sample towards the downstream capture reagent. In some cases, as the sample flows downstream, an analyte (if present) within the sample can bind to the detection reagent (e.g., detection enzyme), where the analyte and the detection reagent may flow towards the downstream capture antibody. In some cases, the analyte and capture antibody may bind and form a complex which may further form an interconnected network (e.g., a precipitate, a lattice). This interconnected network may stop the flow, or significantly reduce the flow rate, of at least some components of the assay, such as the detection reagent(s). Details about this interconnected network are described further below, but briefly, and without wishing to be bound by any particular theory, it is believed that the formation of an interconnected network may result in the formation of a precipitate and/or lattice, in which additional analyte, capture antibody, and optionally the detection reagent, may bind or be trapped (e.g., trapped within pores of the interconnected network). The presence of this interconnected network within the binding region may result in a signal (e.g., a visual signal). In some cases, the signal is visually observable. In some cases, a stain or developing reagent, such as TMB (3,3′,5,5′-tetramethylbenzidine) to be applied to the binding region to generate a signal (e.g., a colorimetric signal). In some instances, during use of such development reagents on the strip may result in backflow of the development reagent and/or sample or other components of the assay (i.e., flow from a downstream to upstream). However, it has also been discovered, as described by this disclosure, that the strip may (at least partially) be enclosed in a cassette that includes a protrusion (e.g., a bridge) extending from a first portion of the cassette to a second, opposing portion of the cassette that may press the strip against the first portion of the cassette, which can reduce or prevent backflow of the sample (or a component within the sample, such as a detection reagent).

Formation of an interconnected network of a plurality of capture reagents (e.g., capture antibodies) and a plurality of analytes is described in more detail below, but briefly, it is believed, without wishing to be bound by theory, that this interconnected network improves the sensitivity (e.g., lowers the limit of detection) of the flow assay compared to certain existing assays in which an analyte binds to a capture reagent but without forming an interconnected network. As described in more detail below, the interconnected network may comprise a precipitate and/or a lattice including a plurality of analytes and/or capture reagents (e.g., a mixture of analytes and capture reagents) wherein at least some of the analytes and/or capture reagents are connected with one another. The connections between the plurality of analytes and/or capture reagents may be a covalent interaction (e.g., a bond) and/or a non-covalent interaction (e.g., ionic interactions, hydrogen bonding, hydrophobic interactions, van der Waals interactions). In some embodiments, the connections between the plurality of analytes and/or capture reagents can be a complementary interaction, such as an antigen-antibody pairing (e.g., one or more antigen-antibody pairings). Other connections are possible. Details regarding the interconnected network within the flow assay are described further below.

Turning to the figures, specific, non-limiting embodiments are described in further detail. It should be understood that the various components, features, systems, assays, and methods described relative to these embodiments may be used either individually and/or in any desired combination as this disclosure is not limited to only the specific embodiments described herein.

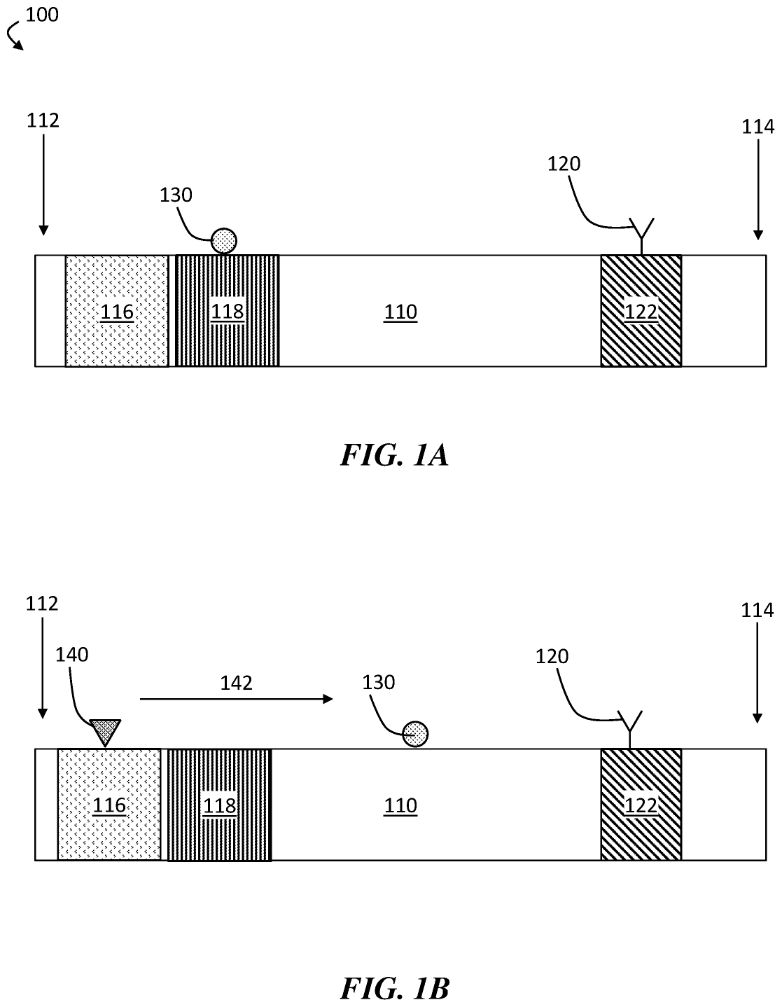

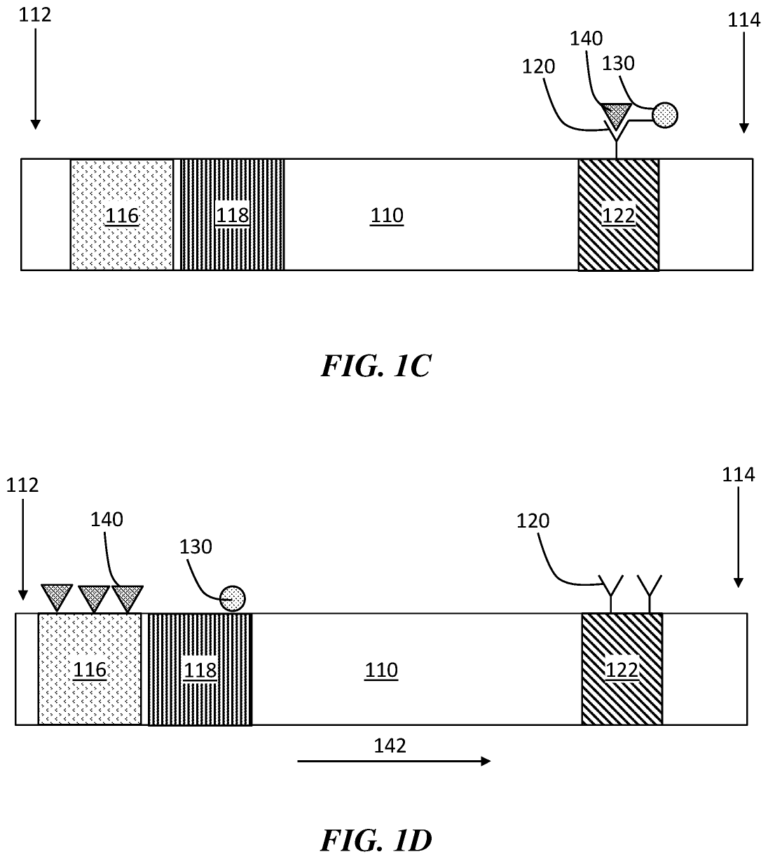

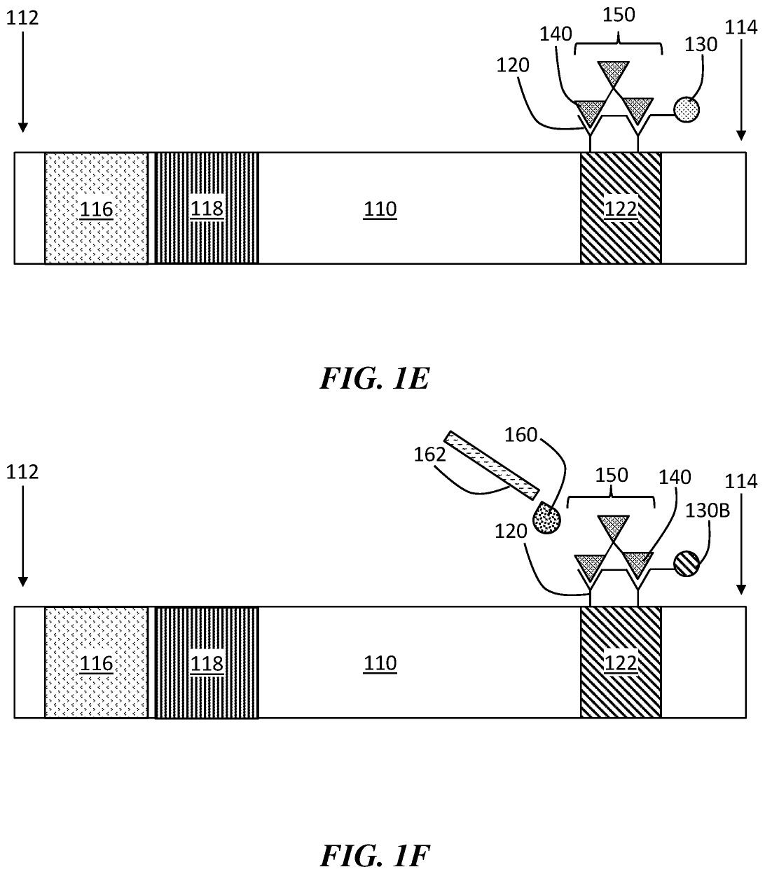

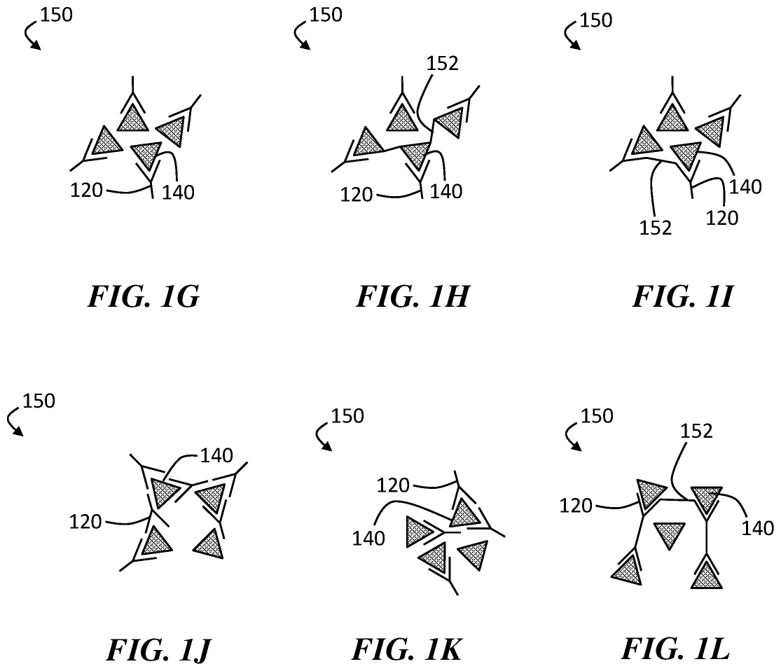

FIG. 1A depicts a schematic diagram of a flow assay 100 used for the detection of an analyte. In the figure, the flow assay 100 includes a substrate 110 comprising a surface with an upstream position 112 and a downstream position 114. Located near the upstream position 112 on the substrate 110 is a sample region 116 and a detection reagent region 118, where the sample and detection reagent(s), respectively, can be deposited. When introducing the sample onto the substrate (e.g., in the sample region), flow of the sample (or an analyte within the sample) flows from an upstream position to a downstream position (e.g., via capillary action, wicking).

The flow assay 100 may also include a detection reagent 130, deposited in the detection reagent region 118. A capture reagent 120, which may be configured to bind to the detection reagent 130 and/or an analyte within the sample, may be positioned at a binding region 122. Accordingly, in some cases, the capture reagent 120 is configured to bind to a particular analyte of interest and may have a size, shape, and/or composition complementary to the detection reagent in order to facilitate binding with both the detection reagent and the analyte of interest.

In FIG. 1B, a sample comprising an analyte 140 has been deposited at the sample region 116, i.e., at a position upstream from the binding region 122. The detection reagent 130 and the analyte 140 may flow (e.g., via wicking) towards the downstream position 114 towards binding region 122, shown as a flow 142 in the figure. First, the detection reagent 130 may bind to the capture reagent 120. Next, after the analyte 140 flows towards the binding region 122, the analyte 140 may bind to capture reagent 120, as schematically shown in FIG. 1C. Upon binding, the analyte-capture reagent-detection reagent complex may be detected (e.g., visually by an external user). Once capture reagent 120 binds the analyte 140, the capture reagent 120 may be configured to remain (e.g., not move) from the binding region 122 (e.g., even as flow of continues from the upstream to the downstream position), as illustrated schematically in the figure.

In some embodiments, a plurality of analytes is present within a sample, and the flow assay may detect one or more of the plurality of analytes. For example, as illustrated schematically in FIG. 1D, a plurality of analytes 140 is present within the sample region 116 and a plurality of capture reagents 120 is present within the binding 122. Flow may be initiated (e.g., flow 142) and the plurality of analytes 140 and the detection reagent 130 may flow towards the plurality of capture reagents 120 (e.g., from an upstream position to a downstream position), as shown in the figure.

The plurality of analytes and/or capture reagent may form an interconnected network (or be configured to form an interconnected network) comprising at least some of the plurality of analytes, capture reagents, and/or detection reagents. For example, as shown in FIG. 1E, an interconnected network 150 including a plurality of analytes and a plurality of capture reagents is shown. Advantageously, formation of the interconnected network may improve the sensitivity of the flow assay compared to certain existing flow assays. Details regarding the interconnected network are described further below.

After formation of the interconnected network, the analyte may be detected. For example, as shown in FIG. 1F, a staining reagent 160 is dispensed from a pipette 162 on to the binding region 122, which includes the interconnected network 150. The staining reagent may change the detection reagent and/or analyte so that the reagent generates a signal if the analyte is present. For example, in the figure, the detection reagent 130B, which may bind to or simply be blocked or trapped by the interconnected network, may generate a signal indicating the presence of at least one analyte 140 and exposure to the staining reagent 160.

The interconnected network (e.g., precipitate, lattice) may have a variety of configurations. FIGS. 1G-1K schematically depict some exemplary configurations. For example, in FIG. 1G, the interconnected network 150 comprises a plurality of capture reagents 120 and a plurality of analytes 140 and are connected by non-covalent interactions between the plurality of analytes 140. In contrast, FIG. 1H depicts the interconnected network joined by at least one bond 152 between two analytes 140. FIG. 1I schematically depicts a configuration for the interconnected network 150 where the bond 152 is between two capture reagents 120. In FIG. 1J, the capture reagent 120 bridges two analytes forming an agglutinate for the interconnected network 150. And FIG. 1K schematically depicts a configuration of the interconnect network 150 in which the analyte 140 bridges two capture reagents 120 forming an agglutinate for the interconnected network 150. In some embodiments, the capture reagent is configured to bind two or more analytes. For example, FIG. 1L schematically shows capture reagent 120 configured to bind two analytes 140, such that the interconnected network 150 comprises the capture reagent 120 binding two analytes. In some embodiments, at least some of the analytes may bind more than one capture reagent and the at least some capture reagents may also bind more than one analyte and form a chain or agglutination of capture reagents and analytes. That is so say, in some embodiments, the interconnected network comprises a chain or agglutination comprising a plurality of capture reagents and/or analytes. Of course, other configurations of the interconnected network are possible, and the configurations are not limited to those schematically depicted in the figures. More details regarding the interconnected network are described below and elsewhere herein.

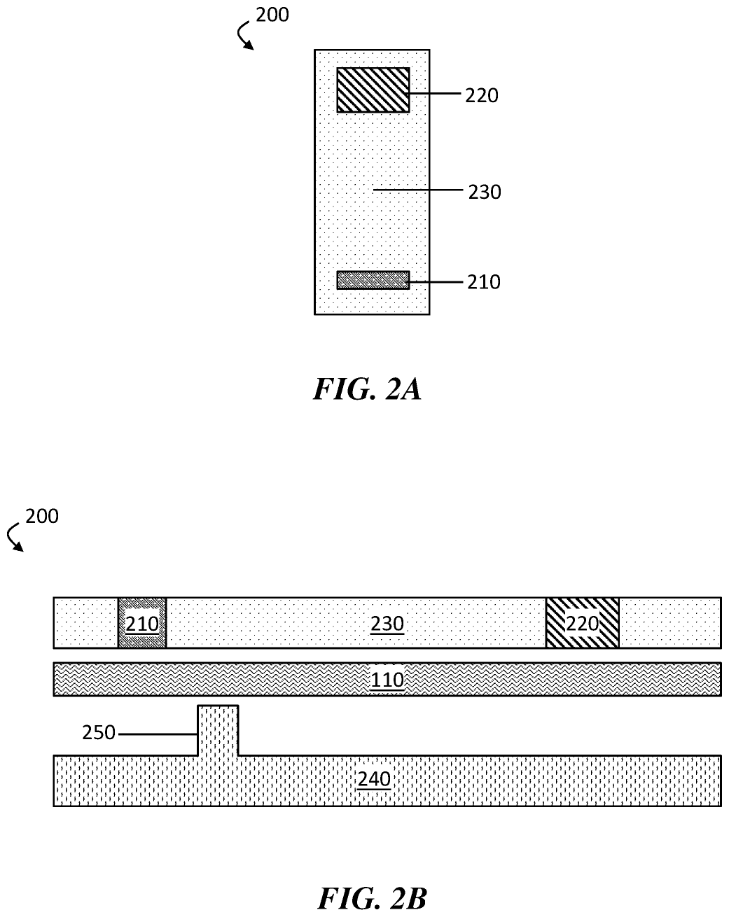

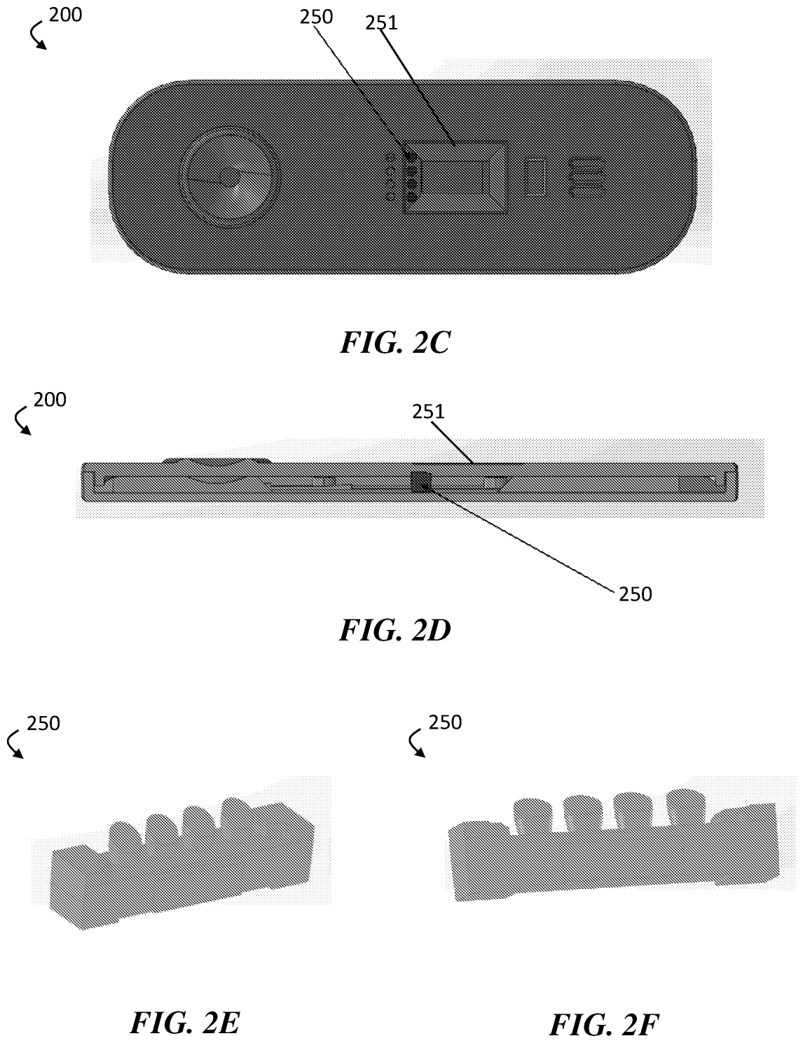

The formation of an interconnected network (e.g., a lattice) within the flow assay may affect the flow properties of the assay. For example, the interconnected network may block pores of the substrate, so that flow is reduced, restricted, and/or stopped on at least some portions of the substrate. In some embodiments, backflow (i.e., flow from a downstream position to an upstream position) is possible. In order to mitigate or eliminate backflow, it was discovered that the substrate can be enclosed (e.g., at least partially enclosed) in a cassette comprising a protrusion that presses the substrate against the cassette. By way of illustration (and not limitation), FIG. 2A and FIG. 2B depict schematic diagrams of such a cassette. FIG. 2A shows a top view of a cassette 200 with a sample window 210 for depositing a sample and a viewing window 220 for viewing the binding region of a substrate. FIG. 2B depicts a cross-sectional view of the cassette 200, showing a first portion 230 and a second portion 240 of the cassette 200, which has a protrusion 250 extending towards the opposing first portion 230 of the cassette. The protrusion may press the substrate against the first portion of the cassette, which may advantageously reduce or eliminate backflow of one or more components of a flow assay (e.g., of a detection reagent, of a sample solvent). And while FIGS. 2A-2B depict one protrusion of the cassette, it should be understood that one or more protrusions may be present. Additional details regarding the cassette and its protrusion(s) are described further below and elsewhere herein.

As mentioned above, the articles and methods described herein may be used for flow assays. For example, in some embodiments, the flow assay is a lateral flow assay. As understood by those skilled in the art, a lateral flow assay is a diagnostic technique to confirm the presence (or absence) of an analyte. Lateral flow assays typically include a planar substrate, such as a sheet or a strip of a material (e.g., paper) that can absorb a liquid sample, and may include a test line where a user can view the results of the assay and a control line so the user can ensure the assay is functioning properly. A sample may be deposited at a sample depositing region at an upstream position of the lateral flow assay and the sample can then flow from an upstream position of the substrate towards a downstream position of the substrate (e.g., towards the test line and/or control line of the substrate). However, it should be noted that while the articles and methods described herein may be suitable for lateral flow assays, other types of flow assays are possible. For example, flow assays may also include chromatographic techniques (e.g., thin layer chromatography, liquid chromatography, reverse phase chromatography), and the sample (or an analyte within the sample) may flow through one or more stationary phases of a chromatography-based assay with the assistance of one or more mobile phases (e.g., solvents) as this disclosure is not so limited. Gel assays or microfluidic channel assays are also possible.

For some embodiments, the substrate of the flow assay has an upstream position and a downstream position. In some embodiments, the substrate comprises a detection reagent (or a plurality of detection reagents) positioned at an upstream position of the substrate. In some embodiments, a sample is introduced to an upstream position of the substrate (e.g., within a sample loading region or within a sample deposition region).

The substrate may comprise any suitable material. In an exemplary embodiment, the substrate is or comprises cellulose (e.g., nitrocellulose). However, other materials are possible. For example, in some embodiments, the substrate comprises or is formed of a polymer or a polymeric material (e.g., cellulose fibers, nylon, PVDF, a polymer gel), without limitation. Other non-limiting examples of substrate materials include paper, glass, quartz, capillary tubes, gels, packed beads (e.g., silica beads), and woven meshes. In some embodiments, the substrate comprises an absorptive material (e.g., cotton, cellulose fiber, absorption pad). In some embodiments, the substrate is or comprises one or more membranes or membrane materials that may selectively pass certain species while rejecting other species (e.g., not permitting an interconnected network comprising one or more analytes to pass while allowing other components to pass). In some embodiments, the substrate includes a flexible or hard material to facilitate easy handling of the substrate. In some embodiments, the substrate comprises one or more portions of layers. In some such embodiments, each portion and/or layer may independently include or be formed of a suitable material.

In some embodiments, the substrate is a porous substrate. For example, in some embodiments, the substrate has a porosity greater than or equal to 20%, greater than or equal to 25%, greater than or equal to 30%, greater than or equal to 40%, or greater than or equal to 50%. In some embodiments, the porosity of the substrate is less than or equal to 70%, less than or equal to 60%, less than or equal to 50%, less than or equal to 40%, less than or equal to 30%, less than or equal 25%, or less than or equal to 20%. Combinations of the above-referenced ranges are also possible (e.g., greater than or equal to 20% and less than or equal to 40%). Other ranges are possible.

In some embodiments, a porous substrate may have pores of a particular pore size. For example, in some embodiments, an average pore size of the pores of a substrate is greater than or equal to 1 μm, greater than or equal to 2 μm, greater than or equal to 5 μm, greater than or equal to 10 μm, greater than or equal to 15 μm, greater than or equal to 20 μm, greater than or equal to 25 μm, greater than or equal to 30 μm, greater than or equal to 40 μm, or greater than or equal to 50 μm. In some embodiments, an average pore size of the pores of the substrate is less than or equal to 50 μm, less than or equal to 40 μm, less than or equal to 30 μm, less than or equal to 25 μm, less than or equal to 20 μm, less than or equal to 15 μm, less than or equal to 10 μm, less than or equal to 5 μm, less than or equal to 2 μm, or less than or equal to 1 μm. Combinations of the above-referenced ranges are also possible (e.g., greater than or equal to 1 μm or less than or equal to 50 μm). Other ranges are possible.

In some embodiments, the substrate comprises one or more layers of materials (e.g., pads of material) that can be stacked on top of one another and/or one or more layers placed adjacent to one another to form the substrate. Retention and/or rejection can be selected based on a variety of factors, including but not limited to, size, charge, and/or mass of a species (e.g., of an analyte, of an analyte-capture antibody complex, of an interconnected network comprising one or more analytes). For example, in some embodiments, the substrate comprises a membrane that may allow some species to pass through while retaining other species based on size exclusion. In some embodiments, the particular pore size, charge, or other properties of the substrate may be important in determining the difference of flow between the free analyte and the analyte within an interconnected network.

It is also noted that, as used herein, when a portion or a layer of a substrate is referred to as being “adjacent” to another portion or layer, it can be directly adjacent to the portion or layer, or one or more intervening components (e.g., portions, layers including, but not limited to, a membrane, a pad, a polymer layer, a glass layer, a coating, and/or a fluid) also may be present. A portion or layer that is “directly adjacent” to another portion of layer means that no intervening component is present.

The substrate or layers of the substrate may have any suitable thickness. In some embodiments, the substrate (or a layer of the substrate) has a thickness of greater than or equal to 100 μm, greater than or equal to 250 μm, greater than or equal to 500 μm, greater than or equal to 750 μm, greater than or equal to 1 mm, greater than or equal to 2 mm, greater than or equal to 3 mm, greater than or equal to 4 mm, greater than or equal to 5 mm, or greater than or equal to 10 mm In some embodiments, the substrate (or a layer of the substrate) has a thickness of less than or equal to 10 mm, less than or equal to 5 mm, less than or equal to 4 mm, less than or equal to 3 mm, less than or equal to 2 mm, less than or equal to 1 mm, less than or equal to 750 μm, less than or equal to 500 μm, less than or equal to 250 μm, or less than or equal to 100 μm. Combinations of the above-referenced ranges are also possible (e.g., greater than or equal to 100 μm and less than or equal to 5 mm) Other ranges are possible.

In some embodiments, a binding region is positioned on the substrate. The binding region may comprise a test line where capture reagents (e.g., capture antibodies) may be deposited. In some embodiments, the binding region also comprises a control line, in which a control reagent (e.g., another capture antibody) may be used in order to ensure the flow assay adequately gives a signal to a user. In some embodiments, the binding region comprising a plurality of capture reagents positioned at the downstream position. In some embodiments, an interconnected networking comprising one or more analytes is deposited before or within the binding region.

In some embodiments, the binding region comprises one or more capture reagents wherein the capture reagent is a single type of capture antibody positioned at or on a surface of the substrate at the binding region. That is, the capture antibody of the single type may bind a single, specific antigen and may be the only type of antibody present within the lateral flow assay (i.e., within the binding region of the flow assay, or within all of the reagents used for the assay for a particular analyte of interest). In some embodiments there may be more than one capture reagent, but these may be of the same type of the single type of capture reagent where each target the same analyte). In some such embodiments, the binding region includes capture antibodies only of the single type such that all the capture antibodies each can bind an analyte, each at the same position of the antibody. However, other embodiments, there may be more than one capture reagent, but each may target a different analyte, or a different portion of the same analyte (e.g., wherein the capture reagent(s) comprise polyclonal antibodies, which each may target a different epitope of the analyte or antigen).

The articles and methods described herein may be used to detect an analyte within a sample. The sample may be any suitable sample containing an analyte of interest or suspected of containing the analyte of interest. As such, in some cases, the sample (or at least a portion of the sample) does not contain the analyte but may be suspected of containing an analyte of interest.

The sample may be obtained from any suitable subject, such as a human or an animal, or may obtained from the environment for environmental testing of analyte (e.g., detection of an analyte in sewage or river water). In some embodiments, the sample is obtained from a cell (e.g., a human cell). In some embodiments, the sample can be obtained from a nasal swab of a patient in order to determine if the patient is afflicted with a disease. In some embodiments, the sample is (or is obtained from) a blood sample, a saliva sample, or a urine sample. In some embodiments, the sample may be processed (e.g., physically processed, chemically processed) prior to its introduction to a sample region in order to (at least partially) purify the sample and/or analyte. For example, the analyte may be contained in a solid sample (e.g., a stool sample) and the solid sample may be further processed (e.g., suspended in a solvent, filtered) to produce a liquid sample.

In some embodiments, the sample may include a solvent for dissolving and/or suspending sample components (e.g., an analyte contained within the sample). In some embodiments, the sample solvent may also facilitate flow of the sample along the flow assay (e.g., via capillary action, via wicking). In some embodiments, the solvent may also be used to wash the flow assay, for example, to wash unbound detection reagents and/or capture reagents from the assay. In some embodiments, the solvent is an aqueous solvent (e.g., water). However, other solvents are possible. The solvent can be selected based on a variety of factors including, but not limited, the ability to dissolve sample components and compatibility with substrate materials of the substrate. Non-limiting examples of other solvents include acetone, acetonitrile (MeCN), benzene, butanol, carbon tetrachloride, chloroform, dichloromethane (DCM), dimethyl formamide (DMF), dimethylsulfoxide (DMSO), dioxane, ethyl acetate, diethyl ether, isopropyl alcohol, ethyl alcohol, methanol, tetrahydrofuran (THF), toluene, or water. Other solvents are possible.

In some embodiments, the sample (or the solvent of a sample) flows via wicking, such as in a lateral flow assay. However, the sample may also flow via other mechanisms, such as via capillary forces or via an applied negative or positive pressure (e.g., via vacuum, via pump, using a compressed gas). In some embodiments, the flow assay is operatively associated with an external pump to provide flow.

The articles and methods described herein may be suitable for detecting a variety of analytes. In an exemplary embodiment, the sample contains an analyte that is or is derived from a pathogen, such as an antigen (e.g., a viral protein or nucleic acid). In some such embodiments, the analyte comprises nucleic acid from or related to SARS-CoV-2. However, other analytes or antigens are possible. In some embodiments, the analyte comprises proteins, peptides, hormones, toxins, nucleic acids (e.g., nucleic acid fragments), and/or gene fragments, or some other appropriate biomarker. In some embodiments, the analyte may be derived from a virus, a bacterium, a fungus, a plant cell, or an animal cell (e.g., a human cell). In some embodiments, the sample is obtained from a fluid sample of a user or a patient (e.g., blood, urine, or saliva).

In some embodiments, the analyte may be detected at a relatively low limit of detection (LOD) compared to certain existing lateral flow assays. In cases where the analyte is derived from a pathogen, the LOD can be determined by measuring the Median Tissue Culture Infectious Dose (TCID50) assay. As understood by those skilled in the art, this assay is performed by adding a serial dilution of a sample containing the pathogen of interest (e.g., a virus) to a plurality of cells in a 96 well plate format. The type of cell is specifically selected to show a cytopathic effect, i.e., a morphological change upon infection of the cells by the pathogen of interest or, alternatively, cell death. After an incubation period, the cells are inspected for CPE or cell death and each well is classified as infected or not infected. In some cases, a colorimetric or fluorometric readout can assist with this classification, which may increase assay sensitivity. The dilution at which 50% of the wells show a CPE or cell death is used to calculate the TCID₅₀. In some embodiments, the concentration of pathogen is less than or equal to 1,000 TCID₅₀/mL, less than or equal to 200 TCID₅₀/mL less than or equal to 32 TCID₅₀/mL, less than or equal to 24 TCID₅₀/mL, less than or equal to 20 TCID₅₀/mL, less than or equal to 15 TCID₅₀/mL, less than or equal to 10 TCID₅₀/mL, less than or equal to 5 TCID₅₀/mL, less than or equal to 3 TCID₅₀/mL, less than or equal to 1 TCID₅₀/mL, less than or equal to 0.5 TCID₅₀/mL, less than or equal to 0.4 TCID₅₀/mL, less than or equal to 0.3 TCID₅₀/mL, 0.2 TCID₅₀/mL or less than or equal to 0.1 TCID₅₀/mL. In some embodiments, the TC50 is greater than or equal to 0.1 TCID₅₀/mL, greater than or equal to 0.5 TCID₅₀/mL, greater than or equal to 1 TCID₅₀/mL, greater than or equal to 3 TCID₅₀/mL, greater than or equal to 5 TCID₅₀/mL, greater than or equal to 10 TCID₅₀/mL, greater than or equal to 15 TCID₅₀/mL, greater than or equal to 20 TCID₅₀/mL, greater than or equal to 24 TCID₅₀/mL, greater than or equal to 32 TCID₅₀/mL, great than or equal to 200 TCID₅₀/mL , or greater than or equal to 1,000 TCID₅₀/mL. Combinations of the above-referenced ranges are also possible (e.g., greater than or equal to 0.1 TCID₅₀/mL and less than or equal to 32 TCID₅₀/mL.). Other ranges are possible.

As mentioned above, the flow assays described herein may include one or more capture reagents (e.g., a plurality of capture reagents). The capture reagents may be configured to bind to an analyte and may also be configured to bind to one or more detection reagents. In some embodiments, each of the plurality of capture reagents may, independently, bind to one or more analytes (e.g., two analytes, three analytes). In some embodiments, a plurality of capture reagents is positioned at a downstream position on a surface of the substrate. In some embodiments, a method comprises allowing a plurality of analytes (or at least a portion of the plurality of analytes) of the sample to bind with at least some of the plurality of capture reagents. In some embodiments, the plurality of capture reagents are configured to form an interconnected network with a plurality of analytes and/or a plurality of detection, or some other capture reagents.

In some embodiments, the capture reagent (e.g., a plurality of capture reagents) is positioned (e.g., immobilized, bound, bonded) on the substrate, for example, within a binding region of the substrate and/or positioned on a test line (e.g., within the binding region) of the substrate. The capture reagent may be positioned on the substrate in a variety of ways, such as via a covalent interaction or via a non-covalent interaction (e.g., absorbed, adsorbed, electrostatic interactions, dispersion forces, or combinations thereof). In an exemplary embodiment, the capture reagent is a capture antibody (for example IgG, IgM, IgA, IgD or IgE antibodies) configured to complementarily bind to one or more analytes (e.g., an antigen or a nucleic acid derived from a virus). In an exemplary embodiment, the capture reagent is also configured to form an interconnected network with other capture reagents and/or with analytes and/or detection reagents. However, other capture reagents are possible, as this disclosure is not so limited. In some embodiments, the capture reagent comprises an aptamer (e.g., a protein, a oligonucleotide) configured bind to a specific target molecule.

In some embodiments, the capture reagent may be located, deposited, immobilized, or positioned (e.g., prior to first use) at a downstream position relative to the detection reagent and/or the sample introduction region (e.g., in a binding region positioned downstream the sample introduction region). However, in other embodiments, the capture reagent may be, alternatively or additionally, located together with the detection reagent (e.g., prior to first use).

In some embodiments, the capture reagent may be configured to complementarily bind to both the detection reagent and also the analyte. In some such embodiments, binding to both the detection reagent and the analyte may allow the capture reagent to remain attached to the binding region of the substrate. However, in some embodiments, when the capture reagent binds the detection reagent but not the analyte, the capture reagent may be configured to be removed (e.g., detached, released) from the binding region of the substrate during use when a liquid flows across the binding region. In some embodiments, when the capture reagent binds the detection reagent but not the analyte, the capture reagent does not form an interconnected network, and can be washed or flowed to a different position on the substrate (e.g., passed the binding region, to a waste region).

In some embodiments, the capture reagent may not be bound to the substrate (e.g., prior to first use) but may subsequently bind to or be positioned on the substrate (e.g., at the binding region) when it binds the detection reagent and/or the analyte. In some such embodiments, the capture reagent may form an interconnected network with detection reagents and/or analytes. In some such embodiments, the capture reagent is configured not to flow (or flow at a slower rate) on or in the substrate (e.g., away from the binding region of the substrate, during first use of the flow assay) if the capture reagent is bound to an analyte. That is to say, in some such embodiments, the analyte (and/or a liquid carrying the analyte), when it binds to the capture reagent, may stop flowing or have a reduced flow rate and/or may cause another component of the assay to stop flowing or have a reduced flow rate (e.g., flow of upstream capture and/or detection reagents may be reduced or stopped when the analyte binds to the capture reagent and/or forms a network comprising the analyte along with capture and/or detection reagents). In some embodiments, the flow of capture and/or detection reagents is restricted (e.g., as a result of the capture reagent-analyte interactions) such that more of the capture reagent remains on the substrate. However, for embodiments in which the sample does not comprise the analyte (e.g., the target analyte), or comprises relatively less analyte, less or none of the capture and/or detection reagents have their flow restricted. Thus, when a developing reagent (e.g., TMB) is applied to substrate (e.g., a binding reagent of the substrate), more development of the capture and/or detection reagents occurs relative to when less (or no analyte) is present within the sample. Without wishing to be bound by any particular theory, it is believed that restriction of flow due to the analyte-capture reagent interactions may be stronger when there is more analyte present (and thus more restriction of the flow rate, relative to a sample containing no or less analyte) and this difference may enable quantitative or semi-quantitative measurement of the amount (e.g., concentration) of analyte within the sample. It is believed this is achieved when more analyte is present within the sample, since more analyte leads to more restriction in the flow and hence it becomes easier to detect the analyte (e.g., visually detect the analyte), as it can bind to more capture reagent, and can also bind to more detection reagent, on the substrate than when there is less analyte (or no analyte) in the sample. In some embodiments, this difference in the restriction of flow may be due to formation of an analyte-capture reagent network (e.g., a lattice) when there is more analyte in the sample relative to a sample with less analyte (or free of the analyte). However, it should be understood that the difference in restriction may occur even in the absence of the formation analyte-capture reagent network.

In some embodiments, flow is restricted when a sample comprising the analyte (e.g., at least one analyte, a plurality of analytes) flows on the lateral flow assay (e.g., flows passed and/or binds one or more capture reagents), wherein flow is not restricted when the sample is free of analyte. These events may occur before the sample and/or liquid has reached the end of the substrate (e.g., the furthermost downstream end of the substrate).

In some embodiments, the flow of a sample and/or the flow of a liquid (e.g., a solvent) on the assay may be restricted, reduced, or otherwise slowed, relative to an initial flow rate. By way of illustration, and not limitation, a sample may flow on the assay at a flowrate of 2 mm/sec, and, for example, after at least one analyte of the plurality of analytes binds to a portion of the plurality of capture reagents, the flowrate of the sample may be restricted 1 mm/sec, or slower. Without wishing to be bound by any particular theory, it is believed that the formation of a mesh or network comprising analyte(s) and/or capture reagent may reduce the flow rate of the sample, for example, due to filing of pores on the substrate (i.e., size exclusion). Alternatively, without wishing to be bound by any theory the binding of analyte to capture reagent may block the pores in the nitrocellulose membrane (or other substrate) either due to physical hindrance, charge based hindrance or some other effect. In some embodiments the flow restriction is due to formation of an interconnected network between the analytes, capture reagents (and in some embodiments other constituents of the sample liquid such as salts and surfactants). In other embodiments simply the individual analyte-capture reagents may simply block the flow thus forming a blocking network which is not interconnected. Without wishing to be bound by any particular theory this may be due to blocking of the pores of a nitrocellulose membrane or other substrate due to size, charge or other restrictions. In some embodiments, the flow rate of the sample and/or the flow of a liquid on/in the assay is reduced by at least 0.9, 0.8, 0.6, 0.5, 0.4, 0.2, or 0.1 times the flow rate of the sample or liquid prior to the flow being restricted (e.g., compared to the flow rate of the sample/liquid at an upstream portion of the substrate, such as at any one of regions 112, 116, 118, and/or 110 shown in FIG. 1A). In some embodiments, the flow rate of the sample and/or the flow of a liquid on/in the assay is reduced by less than or equal to 0.1, 0.2, 0.4, 0.5, 0.6, 0.8, or 0.9, times the flow rate of the sample or liquid prior to the flow being restricted (e.g., compared to the flow rate of the sample/liquid at an upstream portion of the substrate, such as at any one of regions 112, 116, 118, and/or 110 shown in FIG. 1A). Combinations of the above-referenced ranges are also possible. These events may occur before the sample and/or liquid has reached the end of the substrate (e.g., the furthermost downstream end of the substrate).

In some embodiments, the flow rate of the sample and/or the flow of a liquid (e.g., a solvent) on/in the assay is reduced by greater than or equal to 0.1 mm/sec, greater than or equal to 0.2 mm/sec, greater than or equal to 0.3 mm/sec, greater than or equal to 0.4 mm/sec, greater than or equal to 0.5 mm/sec, greater than or equal to 1 mm/sec, greater than or equal to 2 mm/sec, greater than or equal to 3 mm/sec or greater than or equal to 5 mm/sec, relative to an initial flow rate of the sample (or liquid) on the lateral flow (e.g., relative to before the flowrate of the sample is restricted). In some embodiments, the flow rate of the sample and/or the flow of a liquid (e.g., a solvent) on/in the assay is reduced by less than or equal to 5 mm/sec, less than or equal to 3 mm/sec, less than or equal to 2 mm/sec, less than or equal to 1 mm/sec, less than or equal to 0.5 mm/sec, less than or equal to 0.4 mm/sec, less than or equal to 0.3 mm/sec, less than or equal to 0.2 mm/sec, or less than or equal to 0.1 mm/sec, relative to an initial flow rate of the sample (or liquid) on the later flow assay (e.g., relative to before the flow rate of the sample is restricted, such as compared to the flow rate of the sample/liquid at an upstream portion of the substrate, such as at any one of regions 112, 116, 118, and/or 110 shown in FIG. 1A). Combinations of the above-referenced ranges are also possible (e.g., greater than or equal to 0.1 mm/sec and less than or equal to 5 mm/sec). Other ranges are possible. For some embodiments, the flow rate of the sample/liquid is slowed, but not stopped (for example, the flowrate is not 0 mm/sec). For some other embodiments, the flow rate of the sample is stopped. These events may occur before the sample and/or liquid has reached the end of the substrate (e.g., the furthermost downstream end of the substrate).

In some embodiments, restriction and/or reduction of a flow rate of a sample/liquid may occur in any one of the flow assays described herein. In some embodiments, the flow assay comprises a substrate having an upstream position and a downstream position, and a binding region comprising a plurality of capture reagents positioned at the downstream position, wherein the plurality of capture reagents is configured to form an interconnected network with a plurality of analytes, the interconnected network comprising a mixture of the plurality of capture reagents and the plurality of analytes interconnected with one another. Other configurations are also possible. The method may involve, for example, introducing the sample to an upstream position of substrate, wherein the substrate comprises a plurality of capture reagents positioned at a binding region at a downstream position, flowing the sample from the upstream position towards the downstream position, allowing at least a portion of the plurality of analytes of the sample to bind with at least a portion of the plurality of capture reagents, restricting a flow along the flow assay after at least a portion of the plurality of analytes of the sample bind with at least a portion of the plurality of capture reagents, and detecting at least one of the analytes. Other steps are also possible.

In some embodiments the capture reagent is positioned on the substrate but is not bound (or bound lightly) to the substrate so that it can flow along the substrate when a liquid (e.g., a solvent) is applied. However, when the capture reagent binds an analyte and/or a detection reagent, the resulting complex may not move (or moves more slowly) along the substrate. In some embodiments, the complex forms or is within an interconnected network, and the interconnected network may not move or may stop within the binding region of the substrate. More details regarding the interconnected network are described below.

In some embodiments, a chemical species, such as a binding entity, may be attached (e.g., bonded) to the capture reagent, for example, to facilitate its attachment to the substrate. In some embodiments, the chemical species is or comprises biotin, a biotinylated derivative, or other suitable binding entity. That is, the capture reagent may be biotinylated, which may facilitate binding of the capture reagent to the substrate (e.g., a binding region on the substrate). In some embodiments, the capture reagent includes, is attached to, or is bonded to a detection reagent, e.g., via attachment to biotin on the capture reagent. However, other binding entities other than biotin are possible, as this disclosure is not so limited.

As mentioned above, various embodiments may also include one or more detection reagents (e.g., a plurality of detection reagents). For example, in some embodiments, a plurality of detection reagents is positioned at an upstream position on a surface of a substrate. The detection reagent, when present, is configured to facilitate detection and/or identification of the analyte. For example, the detection reagent may be configured to allow detection of an analyte via a color change of the detection reagent itself, by facilitating an enzymatic reaction that can be detected, and/or or by allowing the binding of an additional reporter molecule. The detection reagent(s) may be positioned on any suitable portion of the substrate that facilitates detection of an analyte, if present, within a sample. In some embodiments, a plurality of detection reagents is positioned upstream of the plurality of capture reagents. In some embodiments, a method comprises allowing the plurality of analytes (or at least a portion of the plurality of analytes) of a sample to bind with the at least a portion of a plurality of detection reagents. However, in other embodiments, one or more detection reagents is not positioned on the substrate (e.g., before first use of the flow assay) and may be added subsequently to the flow assay (e.g., after the sample deposited on the flow assay).

The detection reagent may be any suitable reagent for generating a signal for determining the presence (or absence) of an analyte. In some embodiments, the detection reagent comprises a nanozyme (i.e., a metal nanoparticle catalysts), such as nanoparticles of Fe₃O₄. In some embodiments the detection reagent comprises gold and/or latex nanoparticles or some other nanoparticles. In some embodiments the detection reagent comprises enzymes, for example glucose oxidase, cholesterol esterase, cholesterol oxidase or horseradish peroxidase, or a conjugate, molecule or polymer thereof. Other detection reagents are possible.

In some embodiments, articles and methods described herein may also include additional reagents for detecting an analyte. For example, some embodiments may include a reagent (e.g., a staining reagent) configured to react with the detection reagent and/or the analyte in order to generate a signal for detecting the analyte, e.g., by staining the analyte, the detection reagent, and/or a product produced by the analyte and/or the detection reagent (e.g., the detection reagent can react with the analyte to produce a product that generates a signal for detection). The stain may be used to qualitatively detect the presence of the analyte. In an exemplary embodiment, 3,3′,5,5′ tetramethylbenzidine (TMB) is used as a stain after the analyte interacts with the detection reagent. However, other reagents are possible. Non-limiting examples of other reagents include OPD (o-phenylenediamine dihydrochloride), ABTS (2,2′-azino-bis(3-ethylbenzothiazoline-6-sulfonic acid), and/or NPP (p-nitrophenyl phosphate, disodium salt). In some embodiments, the additional reagent(s) for analyte detection comprises nanoparticles (e.g., gold nanoparticles, latex nanoparticles) and/or dyes (e.g., luminol, CPD-Star, ABEI). And while some embodiments may facilitate visual detection, in other embodiments, the analyte may be detected using any suitable detection apparatus, including spectroscopic methods that use absorbance or luminescence, or other spectroscopic detection method, which may be used to qualitatively and/or quantitively determine the presence of the analyte. For example, some embodiments may include a detector (e.g., a PMT detector, a PDA detector) in order to detect the analyte. In some embodiments, a digital reader may be attached to the substrate in order to facilitate detection of the analyte.

The time for detection of an analyte may be of any suitable time. In some embodiments, the time of detection is less than or equal to 30 minutes, less than or equal to 20 minutes, less than or equal to 15 minutes, less than or equal to 10 minutes, less than or equal to 6 minutes, less than or equal to 5 minutes, less than or equal to 3 minutes or less than or equal to 1 minute. In some embodiments, a time of detection is greater than or equal to 1 minute, greater than or equal to 5 minutes, greater than or equal to 10 minutes, greater than or equal to 15 minutes, greater than or equal to 20 minutes, or greater than or equal to 30 minutes. Combinations of the above-referenced range are also possible (e.g., greater than or equal to 1 minute and less than or equal to 30 minutes). Other ranges are possible.

As describe above, the analyte and the capture reagent (e.g., a capture antibody) may form (or be configured to form) an interconnected network. The interconnected network may be formed of or comprise a precipitate (e.g., a precipitate comprising a plurality of analytes and a plurality of capture reagents) or a lattice comprising a plurality of analytes and a plurality of capture reagents. The interconnected network may optionally include other reagents such as a plurality of detection reagents and/or other binding entities. In some embodiments, the interconnected network comprises a plurality of capture reagents and analytes, wherein at least some of the capture reagents and/or analytes interconnect via one or more bridges, the bridge comprising a capture reagent and/or an analyte connecting two or more other capture reagents and/or analytes.

Without wishing to be bound by any particular theory, it is believed that the combination of the soluble analyte and the soluble capture reagent (i.e., soluble in one or more components of the sample, such as the solvent sample and/or sample buffer) may result in the formation of lattice containing the two components that is visible to a user. In some cases, an area of precipitate (e.g., a ring of precipitation) forms when soluble analyte and soluble capture reagent meet on the substrate, and this area may form at the equivalence point between the capture reagent and the analytes (e.g., capture antibodies and antigens), wherein an interconnected network forms (or begins to form) when the capture reagent and analytes are at equivalence. In some embodiments, the lattice is soluble (e.g., at least sparingly soluble, at least partially soluble) while within a flow assay. However, in other embodiments, the lattice is insoluble on or within the flow assay. As understood by those skilled in the art, solubility (or insolubility) may vary depending on a sample solvent, a sample buffer, and may also vary with other factors, such a temperature. Without wishing to be bound by any particular theory, a species is insoluble if a solubility product (Kₛₚ) of the species in a particular solvent at 25° C. is less than 10⁻³ (e.g., less than or equal to 10⁻⁴, less than or equal to 10⁵, less than or equal to 10⁻⁶, less than or equal to 10⁻⁹). A species may be sparingly or partially soluble/insoluble if the Kₛₚ for that species in a particular solvent at 25° C. is between 10⁻³ and 10³ (e.g., between 10⁻³ and 1, between 10⁻² and 1, between 0.1 and 1, between 1 and 10, between 1, and 100, between 1 and 10³). Generally, a species is soluble if the Kₛₚ for that species at 25° C. is greater than 10³ (e.g. greater than 10⁴, greater than 10⁵, greater than 10⁶, greater than 10⁹). Combinations of these ranges may be possible. However, those skilled in the art will understand that these boundaries may vary depending on the factors above, and those skilled in the art, in view of this disclosure, will be capable of selecting analytes and/or capture reagents whose complexes have the appropriate solubility (i.e., a solubility that forms a lattice between an upstream and downstream position or within a binding region of the substrate). In some embodiments, the solvent used in determining the Kₛₚ is water. However, other solvents are possible, for example, the sample solvents described elsewhere herein, without limitation.

In some embodiments, a complex between a capture reagent and an analyte is formed (or is configured to form) and may flow downstream to the binding region. In some embodiments, the size of the complex may increase (e.g., the number of capture reagents and/or analyte reagents in the complex may increase) as it flows from an upstream to a downstream position of the assay. In some embodiments, the complex forms an interconnected network including a plurality of capture reagents and a plurality of analyte reagents. The interconnected network (e.g., comprising a precipitate and/or lattice) may increase in size as the interconnected network migrates or flows towards the binding region. Accordingly, in some embodiments, the size of the complex or interconnected network may increase (or decrease) as it moves from an upstream position towards a downstream position or may increase in size within the binding region of the substrate. In other embodiments, reagents and/or complexes flow downstream but do not form an interconnected network until they arrive at the binding region.

Without wishing to be bound by any particular theory, it is believed that an analyte-capture reagent complex (e.g., an antigen-capture antibody complex) may bind additional analytes and/or capture reagents (e.g., covalently bond, bind via non-covalent interactions) as it flows through the flow assay, or the analyte-capture reagent complex may be immobilized within the binding region and bind additional analytes and/or capture reagents as they move towards the binding region to form the interconnected network. As the analyte-capture reagent complex binds additional analytes and/or capture reagents, the interconnected network (comprising the analyte-capture reagent complex) may increase in size. In some embodiments, the analyte-capture reagent complex or interconnected may also bind (or be configured to bind) one or more detection reagents, which may also increase the size of the complex or interconnected network. In some embodiments, increasing the size of the network comprises an addition of a salt or a buffer to the flow assay.

In some embodiments, a method describes forming an interconnected network comprising the plurality of capture reagents, the plurality of detection reagents, and/or the plurality of analytes. In some embodiments, the interconnected network comprises a precipitate or a lattice comprising a plurality of capture reagents, detection reagents, and/or analytes. In some embodiments, the interconnected network comprises agglomerates of the analyte and the capture reagent. In some embodiments, the interconnected network forms a precipitate within the substrate, for example, within a binding region of the substrate. In some embodiments, the plurality of capture reagents and/or the plurality of detection reagents are configured to form an interconnected network with the plurality of analytes at a downstream position of the substrate. In some embodiments, the interconnected network comprises a mixture of the plurality of capture reagents and the plurality of analytes interconnected with one another. In some embodiments, the plurality of capture reagents and the plurality of detection reagents are configured to form the interconnected network with at least some of a plurality of analytes. In some embodiments, the interconnected network comprises a mixture of the plurality of capture reagents, the plurality of detection reagents, and/or the plurality of analytes interconnected with one another.

In some embodiments, the interconnected network comprises one or more connections (e.g., bonds) connecting the components of the interconnected network (e.g., analytes, capture reagents, and/or detection reagents) to one another. In some embodiments, the interconnected network comprises one or more covalent bonds between an analyte, a capture reagent, and/or a detection reagent (e.g., at least some of a plurality of analytes, at least some of a plurality of capture reagents, and/or at least some of a plurality of detection reagents). In some embodiments, the interconnected network comprises one or more non-covalent interactions between an analyte, a capture reagent, and/or a detection reagent. Non-limiting examples of non-covalent interactions include ionic interactions (e.g., salt bridges), hydrogen bonding, Van der Waals interactions, and/or hydrophobic interactions. For example, in some embodiments, the interconnected network comprises hydrogen bonding between the plurality of analytes, the plurality of detection reagents, and/or the plurality of capture reagents.

In some embodiments, one or more salts (or ionic species of the one or more salts) may contribute to the blockage of the substrate flow. In some embodiments, these one or more salts (or ionic species of the one or more salts) may contribute to formation of the interconnected network formation. The different types of salts and ions may come from various sources, for example, the sample, a sample buffer, a sample solvent, the substrate (e.g., metallic components of a nitrocellulose membrane). Salts (or ionic species of the salt) may influence the fragmentation and/or activation of the side chains of an analyte (e.g., analytes comprising amino acids, such as a protein) during its movement on the substrate. In some embodiments, the salts may also form at least a portion of the interconnected network or may surround at least a portion of the interconnected network in conjunction with the binding of analyte to the capture reagent. In some embodiments, the analyte (e.g., an antigen) and capture reagent (e.g., a capture antibody) binds and may form individual “seeds,” or nucleation sites for the interconnected network (e.g., due to antigen-antibody chains forming or agglomeration) and such seeds may form the basis for salts to accumulate or precipitate around the interconnected network, enhancing the network and may further impede the flow on the substrate once the interconnected network has formed. Advantageously, this effect may lead to an even greater signal generated for positive (target analyte containing) samples versus negative samples which do not contain target analytes. Without wishing to be bound by any particular theory, when the analyte binds to the capture reagent there may be a conformational change and this may lead to the obstruction of pore of the substrate, for example, due to the negative or positives charges from salts (e.g., salts of a buffer system).

In some embodiments, a change in the percentage or concentration of salts and/or other ions may lead to the formation of the interconnected network formation. In some such embodiments, the interconnected network may form a solid precipitate, optionally comprising one or more salts (e.g., salts of a buffer), which may block pores of the substrate. In some such embodiments, the interconnected network may precipitate the formation of salt bridges. In some such embodiments, the salt blockage may “seed” the formation of the interconnected network and may form through several routes, without wishing to be bound by any particular theory: (A) The agglomeration of alternating ionic charges leading to lattice formation comprised of the different types of ions, when present (e.g., within a sample buffer, an antibody solution, HRP conjugate solution). The ions may be either positive or negatively charged. Without wishing to be bound by any particular theory, these charges may play a role in exposing, fragmenting, and/or activating of the side chains of the analytes while it moves on the substrate. Binding of analyte to the capture reagent may cause a blockage in the pore. Further, accumulation of the salts at or within the binding region may change the charge distribution of capture reagents and/or analytes within the binding region and there may be agglomeration; (B) The counterions leading to salt formation where one or more components of a buffer and the substrate contributes, respectively, anions and cations. Some substrates may include cationic components, such as Ca²⁺, Mg²⁺, silicon-containing cations, and these ions may interact with ions of a sample buffer (if present), leading to the formation of the interconnected network or salt bridge (on which the interconnected network may form or precipitate); (C) formation of Ca-phosphate where the substrate includes components like Ca²⁺, Mg²⁺, silicon-containing cations. At a higher pH i.e., >7, there is a tendency of interaction of Ca-phosphate which lead to the formation of CaHPO₄. The presence of NaCl may also contributes to the supply of carbonates which facilitates the precipitation formed by Ca-phosphate.

In some embodiments, a sample comprises a salt and/or a buffer (e.g., phosphate buffer, NaH₂PO₄/Na₂HPO₄). In some such embodiments, salts can contribute to formation of the interconnected network or strengthen the interconnected network by forming such structures around the interconnected network. Non-limiting examples of salts include NaCl, KCl, NH₄Cl, KNO₃, Al(NO₃)₃, (NH₄)₂SO₄, Mg₂SO₄, FeCO₃, CaCO₃, FePO₄, KH₂PO₄, Na₂HPO₄, (NH₄)₃PO₄, Pb(CH₃COO)₂, Cu(CH₃COO)₂. Other salts are possible.

In some embodiments, the sample (e.g., a sample solvent, a sample buffer) comprises a surfactant. In some such embodiments, the surfactant may also attach to capture reagents and/or analytes in such a way as to help restrict flow in the substrate. In some embodiments the surfactants may form at least a portion of the interconnected network or may contribute to the formation of the interconnected network. Without wishing to be bound by any particular theory, the analyte (e.g., an antigen) and capture reagent (e.g., a capture antibody) binds, this may form individual “seeds” and such seeds may form the basis for surfactants to accumulate or precipitate around the interconnected network, enhancing the network and further impeding the flow on the substrate. In some embodiments, the surfactant may play a role in exposing, fragmenting, and/or activating of the side chains of the analytes while it moves on the substrate. In some embodiments, the surfactants may play a role in increasing or decreasing the hydrophilicity of capture regents and/or analytes or other parts of the interconnected network, which can cause or enhance network formation.

In some embodiments, this effect may lead to an even greater signal generated for positive (i.e., analyte-containing) samples versus negative samples which do not contain target analytes.

In some embodiments, the surfactant comprises an anionic surfactant, such as ammonium lauryl sulfate, sodium laureth sulfate, sodium lauryl sarcosinate, sodium myreth sulfate, sodium pareth sulfate, sodium dodecyl sulfate, sodium stearte, sodium deoxycholate, α olefin sulfonate, and/or ammonium laureth sulfate, without limitation.

In some embodiments, the surfactant comprises a cationic surfactant, such as In C8-10 alkyl hydroxyethyl dimethylammonium chloride, C8-10 alkylamidodimethyl propylamine, and/or ditallow dimethyl ammonium chloride, without limitation.

In some embodiments, the surfactant comprises a non-ionic surfactant, such as polysorbates (e.g., tween 20), sorbitan esters, alkyl-phenol polyethylenes (e.g. tritons), oligomeric alkyl-ethylene oxides, poly (alkylene-oxide) block copolymers, without limitation. In some embodiments, the surfactants is an amphoteric surfactant.

In some embodiments, a change in the percentage or concentration of surfactants may lead to the formation of the interconnected network formation. In some such embodiments, the interconnected network comprises the surfactant and/or the surfactant surrounds at least a portion of the interconnected network and may block at least some of the pores of a substrate if the substrate is porous.

In some embodiments, surfactants, such as those described above without limitation, may cause a change in the configuration of the analyte (e.g., causing a protein-containing analyte to change from a “closed” to “open” configuration), which may also enable or enhance binding to the capture reagent.

As mentioned above, various embodiments describe a flow assay for the detection of an analyte. Accordingly, a sample (or a sample solvent) may be flowed. In general, flow is from an upstream position to a downstream position. For example, in some embodiments, a method comprises flowing the interconnected network towards a binding region (e.g., a binding region at a downstream position). In some embodiments, a method comprises stopping flow of the interconnected network within the binding region. In some embodiments, a method comprises, reducing or stopping flow on at least a portion of the substrate after forming the interconnected network. In some embodiments, at least some of the plurality of detection reagents is not flowed off the flow assay during any one of the flowing steps, such as after the formation of the interconnected network in the flow assay. However, as noted above, in some embodiments, the formation of an interconnected network (e.g., a precipitate, a lattice) in the flow assay may affect the flow properties of the assay, such that flow from a downstream position towards an upstream position may occur (i.e., backflow). For example, in some embodiments, a method comprises backflowing the interconnect network away from the binding region.

In some embodiments, a control indicator may be used to indicate when the sample has passed through some or all of the substrate. In some embodiments this control indicator can be a dye placed in the downstream region of the substrate or at a downstream position on substrate (for example, in the absorbent or other pad at the downstream position on or near the substrate), so that when the sample liquid passes through the dye, the dye spreads downstream and appears in a region downstream from its original position. The indicator can be any suitable color, such as a red or other color dye placed at a downstream position of the substrate (e.g., the bottom of the absorbent pad). In the case of a red color indicator, this indicator may spread downstream when the sample passes it so that the red color appears in a window in the cassette downstream from the original position of the dye. In some embodiments, the appearance of the red color in the cassette can be used by an operator to decide when to add detection reagents and/or development reagents to the assay. The indicator may be any colorimetric reagent that flows and can be read by eye or with a reading device. In other embodiments, the color indicator comprises a liquid or solid containing fluorescent particles or charged or magnetic particles. Other color indicators are possible. Use of the indicator, can show when the sample (for example a liquid which may contain the sample analyte) has passed through the substrate. This may indicate that the interconnected network has formed, when there are one or more analytes in the sample, and/or indicates that the detection reagent has flowed off the substrate, when there are no target analytes in the sample. Thus, when the indicator shows, then detection reagents may be added and/or development reagents (for example TMB) may be added, which may differentiate the positive (analyte-containing) sample from the negative sample (does not contain the analyte). The indicator may be a preferred alternative to timing of the sample flow since in many existing lateral flow assays or other systems, the sample can flow at different speeds depending on temperature, humidity, and other factors, but in all cases the indicator shows when the sample has flowed passed that position of the substrate.

As mentioned above, a cassette may be used to modify the flow properties of a flow assay (e.g., a lateral flow assay). Accordingly, some embodiments comprise a cassette at least partially enclosing the substrate. As was described above in view of the figures, the cassette comprises a first portion and a second portion opposing the first portion, and the substrate of the flow assay may be positioned between the first and second portions. In some embodiments, the second portion comprises a protrusion extending towards the first portion, and wherein the protrusion presses the substrate against the first portion. Advantageously, the protrusion may prevent excess sample flow on a first portion of the substrate or cassette rather than though the substrate and also reduce or prevent backflow of flow assay components (e.g., of a sample solvent, of at least a portion of a plurality of detection reagents, of at least a portion of a development reagents. The protrusion can press against the substrate so as to make contact with the first portion, but without causing any liquids (e.g., a sample solvent) to be forced out of the substrate. The protrusion should contact the substrate with sufficient force to prevent the flow of liquids through it or the backflow of liquids through it; however, it also should not press into the substrate with too much force, which could force liquids out of the substrate and/or create an indent or trough in the substrate where liquids could accumulate. For example, such a trough may act as a place where development reagents accumulate and so such excess development reagents are not drawn out of the substrate but instead may remain in the trough. Since certain development reagents may develop even in the absence of development reagents, if given sufficient time, and this may lead to generation of what may appear to be positive signal, even in the case where there are no target analytes in the sample, so this can lead to false positive assays. In other embodiments, false negative assays may be generated due to the indent. Therefore, the control of the specific features and fabrication of the protrusion is essential for proper operation. The cassette may also include one or more windows, for example, for sample deposition and/or viewing a signal for analyte detection. For example, in FIGS. 2C-2D, a viewing window 251 is included within the cassette 200. FIGS. 2E-2F show schematic illustrations of the protrusion (e.g., bridge) 250 schematically illustrated in FIGS. 2C-2D.

In some embodiments, the protrusion positioned to align with a particular position of a substrate. For example, in some embodiments, the protrusion is positioned at a midpoint between an upstream position and a downstream position (e.g., a binding region at a downstream position). In some embodiments, protrusion is less than or equal to 10%, less than equal to 20%, less than or equal to 30%, less than or equal to 40%, less than or equal to 50%, less than or equal to 60%, less than or equal to 70%, less than or equal to 80%, or less than or equal to 90% of a distance between an upstream position and a downstream position. By way of illustration and not limitation, if the distance between an upstream position and a downstream position is 3 cm, then the protrusion could be positioned at 50% of this distance, or 1.5 cm, from the upstream position. In some embodiments, the protrusion is greater than or equal to 10%, greater than or equal to 20%, greater than or equal to 30%, greater than or equal to 40%, greater than or equal to 50%, greater than or equal to 60%, greater than or equal to 70%, greater than or equal to 80%, or greater than or equal to 90% of a distance between the upstream position and the downstream position. Combinations of the above-referenced ranges are also possible (e.g., the protrusion is positioned greater than 10% of a distance between the upstream position and the downstream position and less than or equal to 90% of a distance between the upstream position and the downstream position). Other ranges are possible.

The protrusion may extend from a surface of a second portion towards the first portion. The extent of this protrusion may affect the degree of force applied to a substrate, when present, as it presses against the first portion. In some embodiments, the protrusion extends greater than or equal to 0.1 mm, greater than or equal to 0.2 mm, greater than or equal to 0.3 mm, greater than or equal to 0.5 mm, greater than or equal to 0.7 mm, or greater than or equal to 1 mm from a surface of the second portion. In some embodiments, the protrusion extends less than or equal to 1 mm, less than or equal to 0.7 mm, less than or equal to 0.5 mm, less than or equal to 0.3 mm, less than or equal to 0.2 mm, or less than or equal to 0.1 mm from a surface of the second portion. Combinations of the above-referenced ranges are also possible (e.g., the protrusion extends a distance from a surface of the second portion by greater than or equal to 0.1 mm and less than or equal to 1 mm). Other ranges are possible.

In some embodiments, the protrusion is formed of the same material as another part of the cassette, for example plastics, including, but not limited, to polyethylene, polypropylene, ABS, HIPS. In other embodiments, the protrusion is formed from a different material as another part of the cassette.

In some embodiments the protrusion may be formed of a different material from other parts of the cassette. For example, the protrusion may be formed from a softer material relative to other components of the flow assay. In some embodiments, the protrusion comprises a soft material, for example rubber (e.g., silicone rubber) or thermoplastic elastomer (e.g., polyurethane) either forming the whole of the protrusion or over molded onto another material which is the base of the protrusion. In some embodiments, the softer material may have a lower shore of 25-40 A or less. Advantageously, the use of the low shore material is that it may be difficult to achieve exactly the depth required for a hard protrusion into the substrate (for example nitrocellulose membrane) in order to have exactly the right force (i.e., not too much force as to squeeze liquid from the substrate, but not too little force as to provide an inappropriate amount of force for pressing the substrate). Such tight tolerance can be difficult to achieve in mass manufacturing. However, it has been discovered by this disclosure, that the ese of a softer protrusion with shore 25-40 A material instead can improve the manufacturing of the protrusion.

The articles and methods disclosed herein are suitable for a variety of applications. In some embodiments, an article is a flow assay (e.g., a lateral flow assay). As mentioned above, for example, the articles and methods disclosed may be used to detection of pathogens, such as SARS-CoV-2. The articles and methods disclosed may be used to detect other pathogens, such as variants of SARS-CoV-2 or influenza viruses, as non-limiting examples. However, other applications are possible. For example, the articles and methods described herein may be used to test for the presence of an environmental contaminant, such as a pollutant or a toxin suspected of being present in a particular environment.

### Example 1

The following example shows that the movement and location of the capture reagent (antibody) and detection reagent (HRP conjugate) when run with a positive sample (i.e., containing the analyte) and negative sample (i.e., not containing the analyte). Here HRP means horseradish peroxidase and HRP conjugate, means HRP conjugated to some other molecule or species, either covalently or non-covalently.

To test the concept that, in negative sample, the HRP conjugate and biotinylated antibody detection reagent are being washed out into the absorbent pad of the substrate in the case of negative samples and remaining in the nitrocellulose membrane substrate when the target protein is contained in the sample, 6 devices were used, 3 with PCR confirmed positive and 3 with PCR confirmed negative.

The sample was added to the sample pad. Once the sample had flowed through the strip and entered the absorbent pad of the substrate. The absorbent pad was removed and placed into the well of a 96-well plate. 150 μl TMB staining reagent was added. No development was observed for 20 minutes at room temperature.

Since the presence of detection reagent HRP conjugate on the absorbent pad is known, given the flow of the sample, it was considered highly likely that the absorbent pad was preventing the staining reagent TMB from coming into contact with the detection reagent HRP conjugate and/or the majority of the HRP conjugate was on the backing pad of the strip. A second example, Example 2, was designed providing different method to locate the detection reagent HRP conjugate.

### Example 2

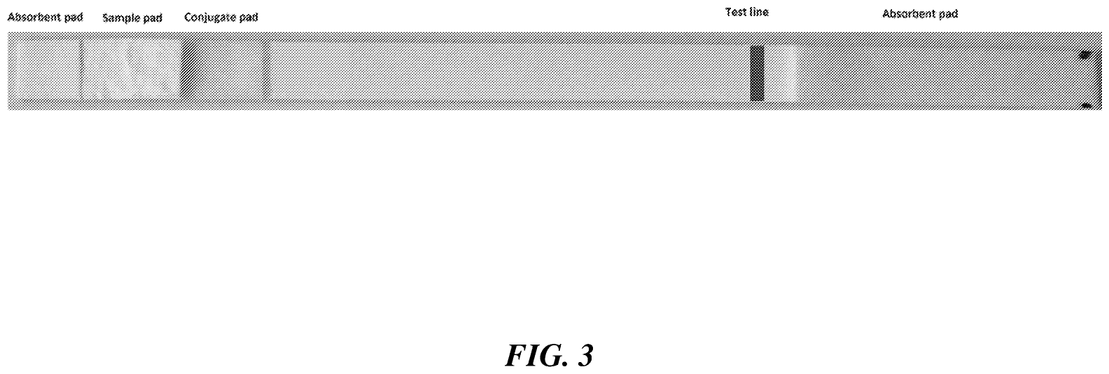

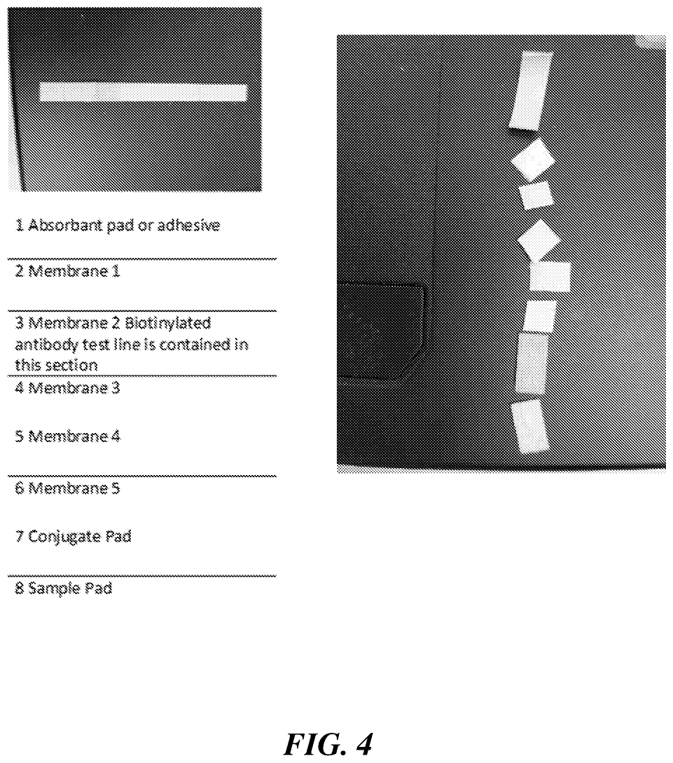

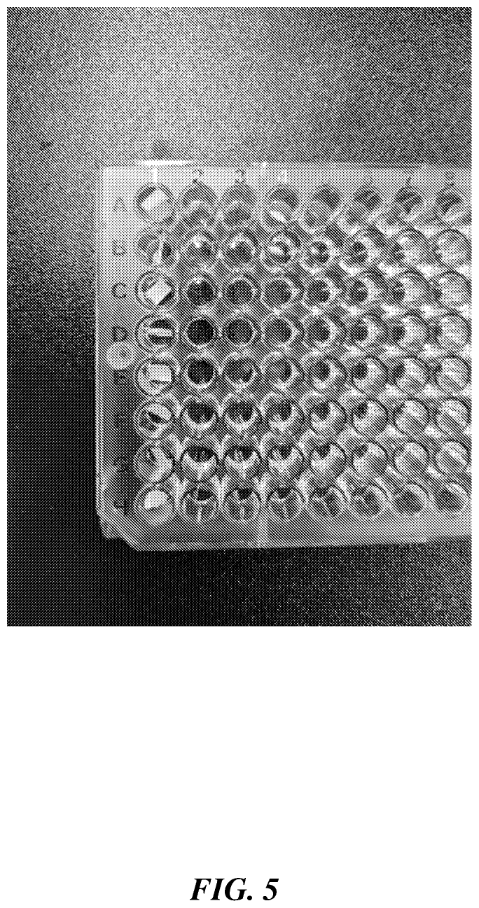

The following example describes isolating each section of the lateral flow assay to determine the presence and amount of the detection reagent HRP conjugate. 12 flow assays were used, 6 with an absorbent pad (FIG. 3) and 6 without. For each device type, 6 samples were run 3 with PCR-confirmed positive samples and 3 samples run with PCR-confirmed negative samples. Sample was added to the sample pad. Once the flow reached the absorbent pad, having flowed through the full membrane substrate, the lateral flow assay was dismantled into its component parts, and the 2.5 cm membrane substrate was cut into 0.5 cm sections, creating 8 discrete regions of the assay, as shown in FIG. 4. Sections were placed into individual wells of a 96-well plate as shown in FIG. 5. 150 μL of staining reagent TMB was added. The reaction was stopped after 17 minutes by adding 150 μl stop solution. The assay components were removed from the wells, and the plate was read at 450 nm.

### Results

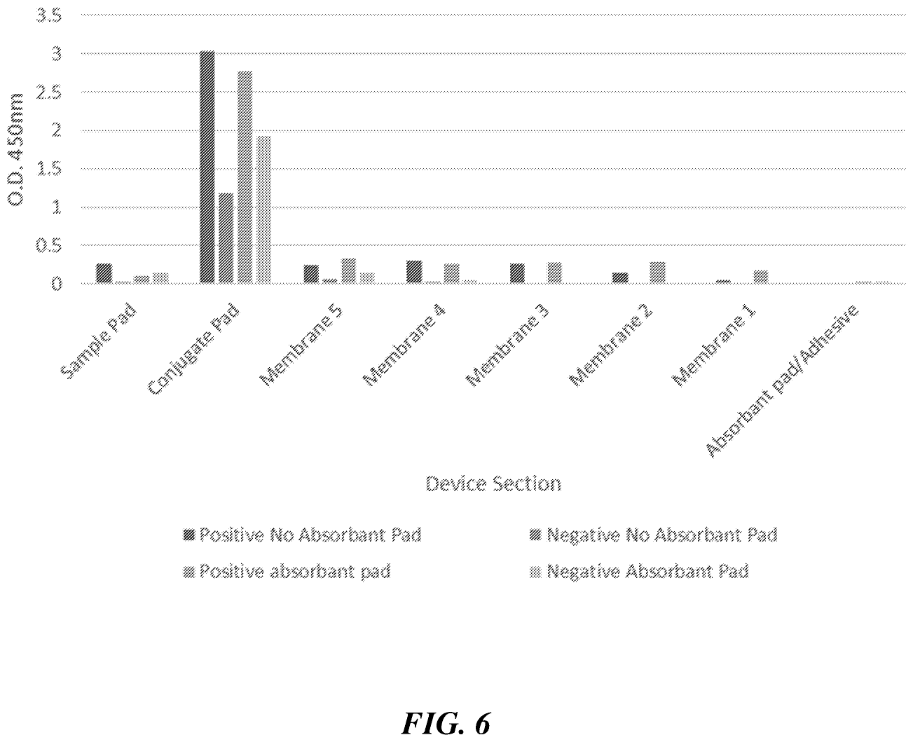

The results are shown in FIG. 6. The detection reagent region conjugate pads in all lateral flow assays still contained significant amount of HRP conjugate. For the assays on which positive sample were run, the level of enzyme remained at a constant level until the next section of the membrane substrate, section 2, which contained the antibody with a lower level past the test line. By contrast, the negative samples had a much lower level of enzyme remaining in the conjugate pad, and very low levels in the 2 sections of membrane following, and negligible levels in membrane sections 1-3. The lower levels of enzyme in the conjugate pads of negative samples, and low levels across the membrane, indicate that the HRP conjugate has been largely washed out of the membrane by the sample in the negative sample (no target antigen/protein) case. This suggested that for the positive samples, flow was reduced or stopped, while for the negative sample, flow was not reduced or stopped, and the HRP conjugate could be largely washed off the assay substrate (membrane).

The presence of high levels of enzyme in each section of the devices run with positive samples, which drops off after the test-line, indicated that the binding of the target analyte antigen to the biotinylated antibody capture reagent has restricted the flow of the sample, preventing the enzyme from flowing through.

The antigen-HRP conjugate complex (network or mesh) appears to be blocking the flow, either through physical blocking of the pores of the substrate or by causing changes to the nature of the membrane substrate (e.g., pH or charge) or any other mechanism, which restrict the complex formed through a cascade or chain reaction between the antigen, antibody and/or HRP conjugate. In the absence of antigen (negative sample), no restrictive complex (network) is being formed and the HRP conjugate is able to be washed away.

### Example 3

The following example describe analyzing the flow pattern of the sample on a lateral flow assay.

In order to further confirm the observation discussed in Example 2, a third example was design with the aim to trace the flow profile of the sample and in order to further analyze the movement of the HRP conjugate detection reagent along the strip (substrate) in the presence and the absence of the antigen (positive and negative samples).

Lateral flow assays were fabricated with longer nitrocellulose membrane substrates compared to the assay of Example 2. An additional 1 cm was added from the test line, leaving the same distance between the sample pad and the test line as in standard devices (strip used in experiment 2), while moving the absorbent pad 1 cm further from the test line.

### Results

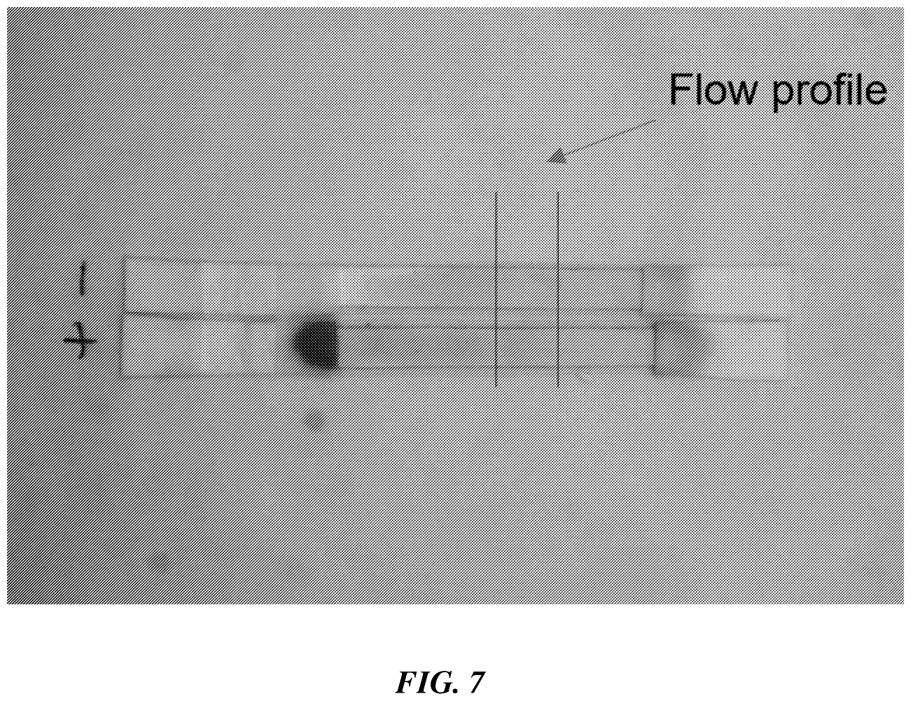

The assays were run as previous described above, with the addition of the TMB staining reagent in the test region once the sample had stopped flowing along the membrane. The TMB staining reagent was added, and the TMB development along the whole strip was generated to trace the flow profile. As shown in FIG. 7, two flow profiles can be distinguished in the test line region, depending on the sample. For the negative sample, a Poiseuille-type flow profile is observed, suggesting a fully developed velocity flow of the negative sample. While for the positive sample, a hydrodynamic entrance region can be distinguished with an irrotational flow region.

This result is consistent with the previous observation in Example 2 and demonstrated that, in the negative sample, the complex will flow along with the sample fluid and reach the absorbent pad. By contrast, in the positive sample, the antibody bound to the target antigen analyte that forms a complex through a cascade or chain reaction between the antigen, antibody capture reagent and/or HRP conjugate detection reagent. The trapped complex creates an obstructed region, resulting in an irrotational flow region.

### Example 4

The following example uses IgG as the detection reagent.

An anti-S1 IgM was biotinylated and used in devices in place of the biotinylated IgG detection reagent. Positive and negative samples were run using these samples.

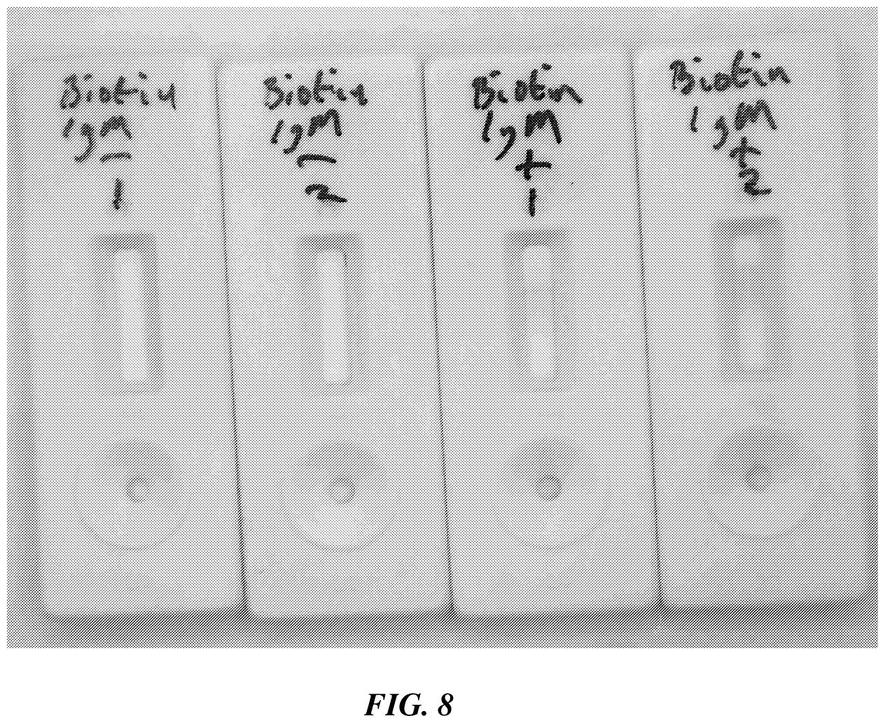

The positive samples gave good development and the negative samples remained blank. The size of IgG is 150 kDa. The results are shown in FIG. 8. By contrast, IgM is 970 kDa. Given the large difference in size between the two types of antibodies, this result suggested that it is not only the size of the antibody-antigen complex that is preventing movement along the membrane substrate, that there is a further interaction, either between a single complex and the membrane substrate, or between the two or more antibody-antigen complexes and/or the salts or other components of the sample buffer. The latter suggests the formation of an interconnected network as the flow assay is run.

### Example 5

The following example demonstrates that other analytes can be used other than the SARS-CoV-2 spike glycoprotein S1 subunit. For this example, Respiratory Syncytial Virus (RSV) was selected as the source of the analyte.

A mouse anti-RSV antibody IgG, which targets the F protein, MAB12398-100 was used as a detection reagent. Respiratory Syncytial virus A lysate was used as a target analyte. Initially, dilutions were made at 1 in 10 from 100 μg/mL down to 1 pg/mL. With positive samples, signal was achieved at 1 pg/mL, and serial 1 in 2 dilutions were made from 0.5 pg/mL down to 3.91 fg/mL in an extraction buffer.

### Results

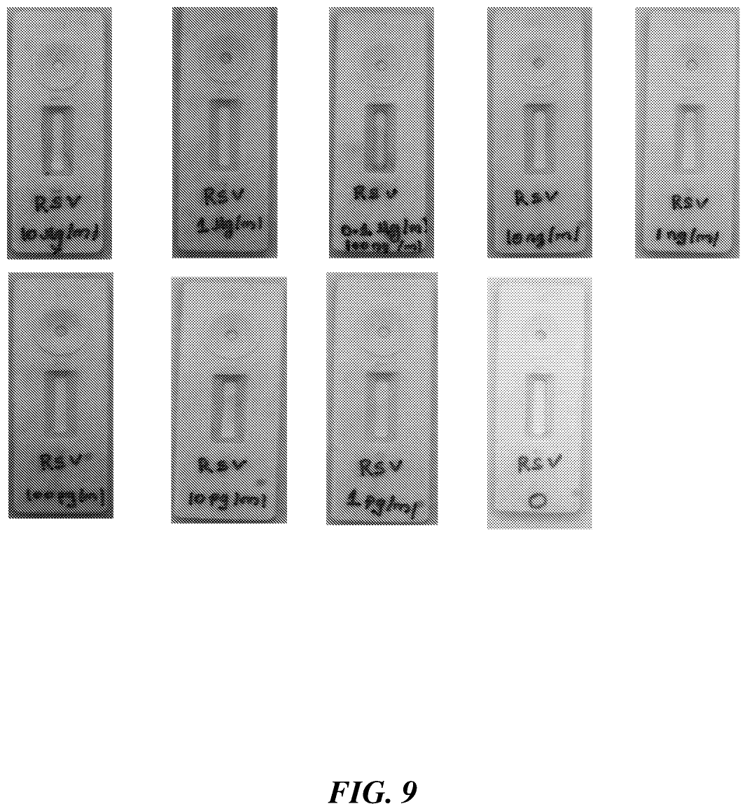

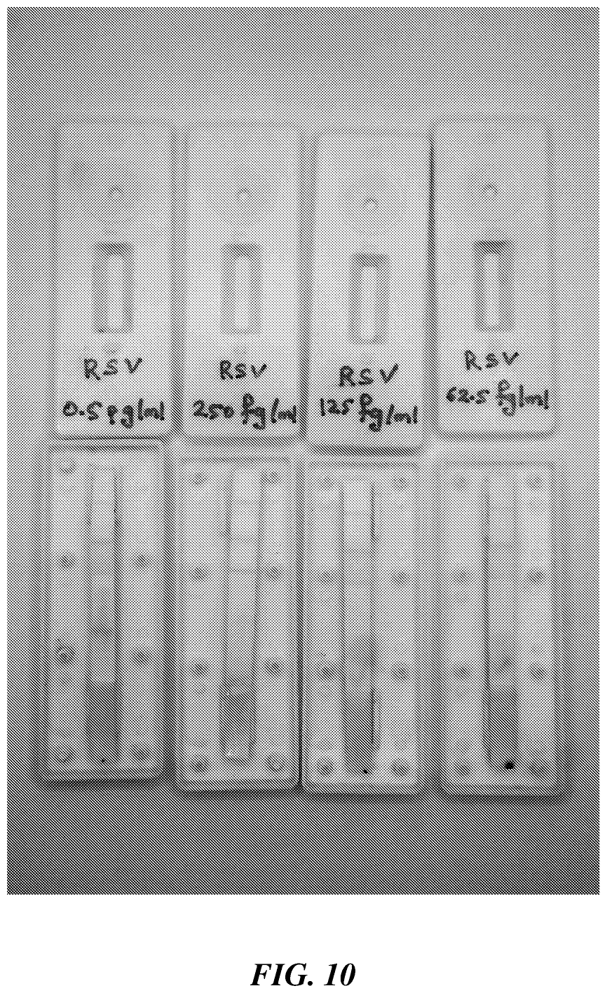

The results of each assay are shown in FIGS. 9-10. As can be seen, RSV lysate gave a positive signal down to 62.5 fg/mL. These results show that the mechanism describe in the previous examples can be used to detect other pathogens and biomarkers, and is not restricted to the SARS-CoV-2 Spike glycoprotein S1.

Similarly, PSA was also run and tested in the same way and showed detection down to pg/mL level and no signal in the negative sample case (no target antigen). Since there can be a large variation in size between these various antigens (PSA is very small for example), this result suggested that it is not only the size of the antibody-antigen complex that is preventing movement along the membrane substrate, that there is a further interaction, either between a single complex and the membrane substrate, or between the two or more antibody-antigen complexes and/or the salts or other components of the sample buffer.

### Example 6

The following example describes determining the position of the capture antibody after running a positive and a negative sample.

To test the concept that the capture antibody is flowing away from the test line in the case of a negative sample and remaining in place in the case of a positive sample, the antibody was labelled with a fluorophore to enable imaging of the strip via microscopy.

Anti-S1 IgG was conjugated with a UV label using a commercially available conjugation kit.

The antibody was deposited at the test line on the nitrocellulose membrane, and test strips were manufactured following a procedure similar to the previous examples.

A SARS-CoV-2 positive nasal swab was run on one device, and a negative nasal swab was run on another. The positive and negative test strips were imaged using a fluorescent microscope from 6 minutes following the application of the sample and imaged at 50 second intervals over a 10 minute period.

### Results

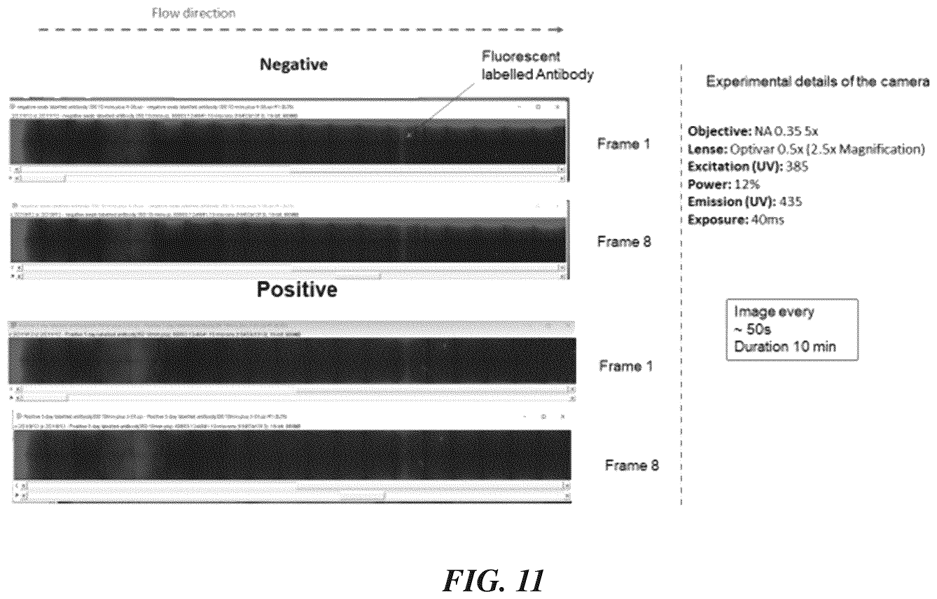

The results are shown in FIG. 11. The antibody line in both positive and negative samples remains in the same position through the 10-minute period of imaging. This indicates that during the manufacturing of the test strip, the antibody binds strongly to the nitrocellulose membrane and does not flow off the test line in the case of positive and negative samples.

### Example 7

The following example describes labeling of streptavidin-linked HRP conjugate to visualize the flow of the enzyme through the test strip during the run of a positive and negative nasal swab sample.

To test the concept that the flow of the enzyme, which acted as a detection reagent, was restricted in the case of positive samples, and not restricted in the case of negative samples the enzyme was labeled to enable time lapse imaging of the labeled enzyme during flow through the membrane .

An ATTo 488 as fluorophore was attached to the enzyme. The streptavidin-linked HRP conjugate was incubated with a biotinylated fluorophore at a 1:4 ratio, in order to bind all the binding sites on the streptavidin.

The fluorophore-conjugated streptavidin-linked HRP conjugate was deposited onto the conjugate pads and test strips manufactured as described elsewhere herein.

A SARS-CoV-2 positive nasal swab was run on one device, and a negative nasal swab was run on another.

The positive and negative test strips were imaged using a fluorescent microscope from 6 minutes following the application of the sample and imaged at 30-second intervals over a 10-minute period.

### Results

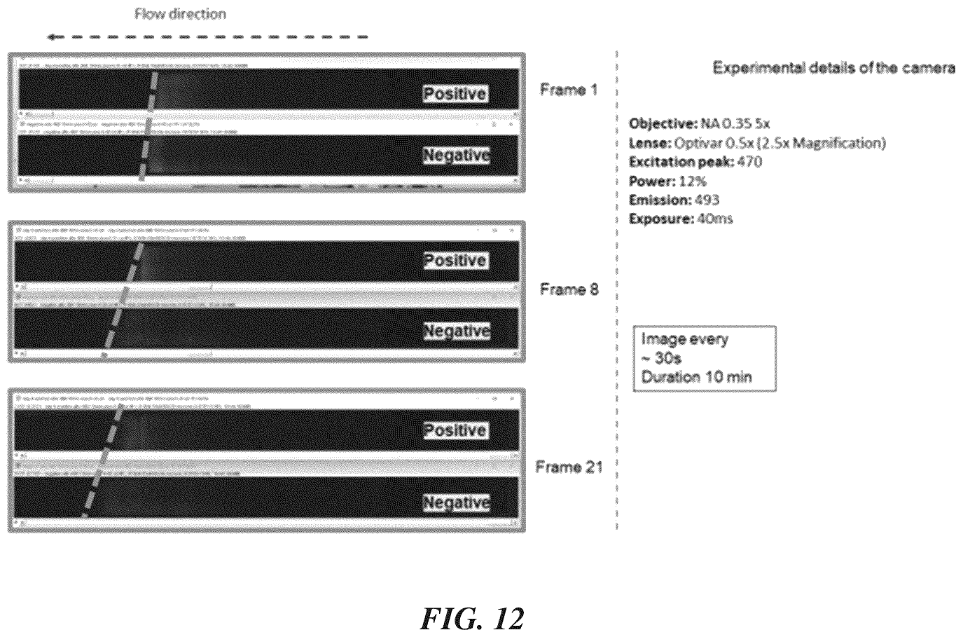

The flow of the enzyme in the positive swab sample is clearly delayed when compared with the negative sample, indicating a restriction of flow. The enzyme becomes more concentrated towards the test line and front end of the absorbance pad in the positive over the 10-minute period of imaging, while in the negative sample, the flow disperses out into the absorbance pad over the 10-minute period of imaging. These results are visualized in FIG. 12.

### Example 8

The following example describes the use of HRP substrate TMB to examine the relationship to enzymatic development with the fluorophore-enabled imaging of the movement of the enzyme, and to determine whether the addition of TMB results in further movement of the enzyme within the test strip.

The streptavidin-linked HRP conjugate was incubated with a biotinylated fluorophore at a 1:4 ratio, in order to bind all the binding sites on the streptavidin.

The fluorophore-conjugated streptavidin linked HRP conjugate was deposited onto the conjugate pads and test strips manufactured as described elsewhere herein.

A SARS-CoV-2 positive nasal swab was run on one later flow assay device, and a negative nasal swab was run on another.

The positive and negative test strips were imaged using a fluorescent microscope from 6 minutes following the application of the sample and imaged at 30 second intervals over a 10-minute period.

At the end of the 10-minute imaging period, the strips were removed from the microscope. TMB was deposited onto the test line and allowed to develop for 5 minutes. The development was imaged, and the test strips were placed back into the microscope to image the final position of the enzyme.

### Results

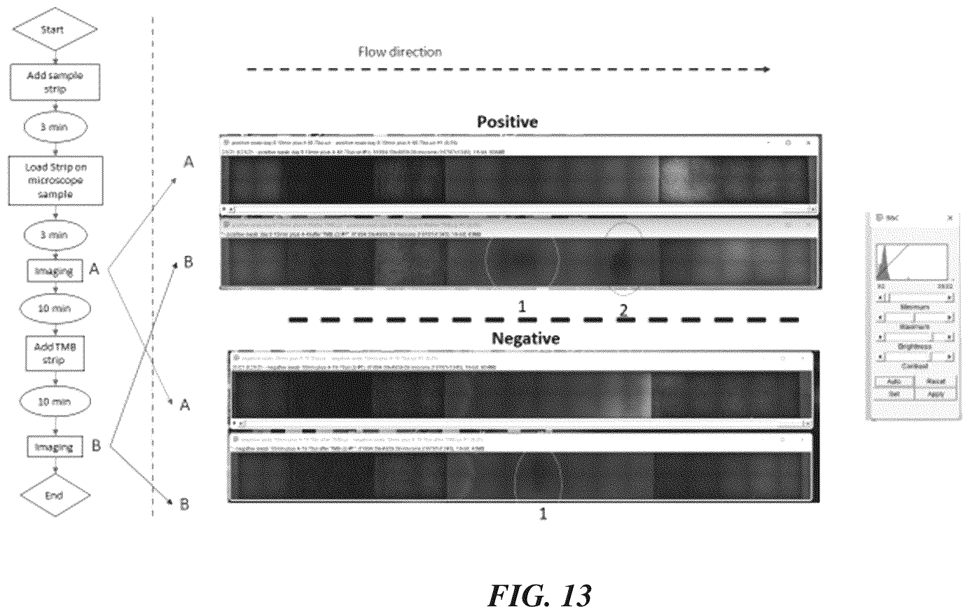

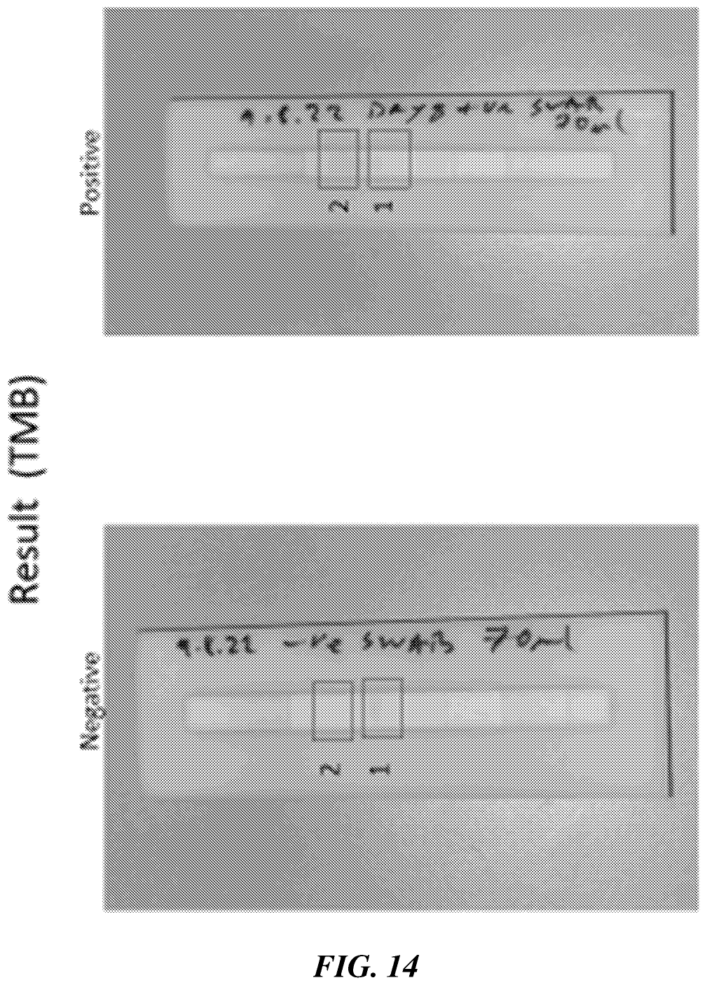

The results are shown in FIG. 13 and FIG. 14 in the case of the negative sample, following the deposition of TMB on the test line, a single area of development occurred upstream of the test line (i.e., region 1 in FIG. 13) as the TMB flowed back along the nitrocellulose membrane, demonstrating the presence of enzyme at this location.

In the case of the positive sample, following the deposition of TMB at the test line, a clear area of strong development occurred adjacent to the test line (i.e., region 2 in FIG. 13), followed by development in region 1, as seen in the case of the negative sample. This indicates 2 distinct regions in which enzyme is present in the case of a positive sample, with the greater amount retained adjacent to the test line.

Fluorescent imaging of the strips following the TMB development revealed a non-specific signal or background (i.e., not related to the enzyme) due to unbound fluorophore all along the nitrocellulose membrane. The figure shows dark regions, which correspond to the regions of TMB development observed (FIG. 14). This may be due to the optical density of the developed TMB restricting the excitation and/or emission of the fluorophore.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for detecting a plurality of analytes in a sample using a flow assay, the method comprising:
introducing the sample to an upstream position of substrate, wherein the substrate comprises a plurality of capture reagents positioned at a binding region at, a downstream position;
flowing the sample from the upstream position towards the downstream position, allowing at least a portion of the plurality of analytes of the sample to bind with at least a portion of the plurality of capture reagents;
flowing towards the downstream position a plurality of detection reagents that are unresponsive in the presence of the antigen;
physically trapping at least a portion of the detection reagents with analytes that are bound to capture reagents; and
detecting trapped detection reagents.

2. The method of claim 1, wherein bound analytes and capture reagents form an interconnected network.

3. The method of claim 2, further comprising increasing a size of the interconnected network.

4. The method of claim 2, further comprising reducing or stopping flow on at least a portion of the substrate after forming the interconnected network.

5. The method of claim 1, wherein a flowrate of the sample is reduced by greater than or equal to 0.1 mm/sec and less than or equal to 5 mm/sec.

6. The method of claim 2, wherein the interconnected network comprises non-covalent interactions between the plurality of analytes, the plurality of detection reagents, and/or the plurality of capture reagents.

7. The method of claim 1, wherein the plurality of detection reagents is positioned at the upstream position on a surface of the substrate.

8. The method of claim 2, wherein the interconnected network forms a precipitate within the substrate.

9. The method of claim 1, wherein the sample includes a salt, a buffer, and/or a surfactant.

10. The method of claim 1, wherein the plurality of capture reagents is a plurality of capture antibodies, and the analyte is an antigen.

11. The method of claim 1, wherein the detection reagent is one or more HRP conjugates.

12. The method of claim 1, wherein the detection reagent is one or more nanoenzymes.
